(19) United States
(12) Patent Application Publication
(10) Pub. No.: US 2022/0389102 A1
(43) Pub. Date: Dec. 8, 2022

(54) HLA CLASS I SEQUENCE DIVERGENCE AND CANCER THERAPY

(71) Applicants: Memorial Sloan Kettering Cancer Center, New York, NY (US); Max-Planck-Gesellschaft zur Förderung der Wissenschaften e.V., Munich (DE)

(72) Inventors: Timothy A. Chan, Cortlandt Manor, NY (US); Diego Chowell Puente, New York, NY (US); Chirag Krishna, Redwood City, CA (US); Tobias Leander Lenz, Hamburg (DE); Federica Pierini, Rome (IT)

(21) Appl. No.: 17/770,259

(22) PCT Filed: Oct. 30, 2020
(86) PCT No.: PCT/US20/58389

§ 371 Date: Apr. 19, 2022

Related U.S. Application Data
(60) Provisional application No. 62/929,756, filed on Nov. 1, 2019.

(51) Int. Cl. C07K 16/28; A61P 35/00

(52) U.S. Cl. CPC C07K 16/2818; C07K 16/2827; A61P 35/00; A61K 2039/505

(57) ABSTRACT

Molecular determinants of cancer response to immunotherapy are described.

### CROSS-REFERENCE TO RELATED APPLICATION

This application claims the benefit of U.S. Provisional Application 62/929,756, filed Nov. 1, 2019, the contents of which are hereby incorporated by reference in their entirety.

### GOVERNMENT SUPPORT

This invention was made with government support under CA232097, CA205426 and CA008748 awarded by the National Institutes of Health. The government has certain rights in the invention.

### BACKGROUND

Cancer immunotherapy involves the attack of cancer cells by a patient's immune system. Regulation and activation of T lymphocytes depends on signaling by the T cell receptor and also cosignaling receptors that deliver positive or negative signals for activation. Immune responses by T cells are controlled by a balance of costimulatory and inhibitory signals, called immune checkpoints. It is predicted that worldwide cancer spending will exceed $150 billion by 2020, in significant part due to immunotherapy drug development.

### SUMMARY

Immunotherapy strategies (i.e., therapeutic regimens and modalities that supplement, enhance, or induce a patient's own immune response, or otherwise harness or stimulate the patient's immune system, to target tumors) have revolutionized the treatment of patients with advanced-stage cancers. Among such strategies are administration of so-called “checkpoint inhibitors”, which are often antibody-based therapeutic agents (e.g., monoclonal antibodies or antigen-binding components thereof) that target an immune checkpoint regulator such as cytotoxic T lymphocyte-associated protein 4 (CTLA-4), and/or programmed cell death protein 1 (PD-1) or its ligand (PD-L1)1. The present disclosure recognizes the source of a problem that can arise with respect to immunotherapy regimens. In particular, it has been observed that durable benefit from these approaches is often limited to a minority of patients.

Recent work has discovered that likelihood of a favorable response to cancer immunotherapy can often be predicted. See, International Patent Application WO2016/081947 to Chan et al of the Memorial Sloan Kettering Cancer Center, incorporated herein by reference. Among the critical determinants of ICI response is tumor mutational burden, a proxy for the number of tumor-derived neoantigens that can be presented on the cell surface by MHC molecules. These neoantigens facilitate anti-tumor immunity through recognition by cytotoxic T-cells4. Another related genetic factor that determines the success of ICI is heterozygosity at the highly polymorphic HLA-I loci1.

The present invention encompasses a discovery that likelihood of a favorable response to cancer immunotherapy for a wide range of different cancers can be predicted through analysis of physiochemical and protein sequence divergence between HLA-I alleles of a patient's genotype.

In some embodiments, the present disclosure provides a method comprising steps of: administering immune checkpoint inhibitor therapy to a subject suffering from cancer who has a high HLA-I evolutionary divergence (HED). In some embodiments, the present disclosure provides a method comprising steps of: determining a HLA-I evolutionary divergence (HED) of a subject suffering from cancer; identifying a subject with a high HED as a candidate for treatment with an immunotherapy. In some embodiments, the present disclosure provides a method of treating a subject suffering from cancer comprising: determining that the subject has a high HLA-I evolutionary divergence (HED); administering immunotherapy.

In some embodiments, the present disclosure provides a method of determining if a subject suffering from cancer will respond to immunotherapy comprising determining the subjects HLA-I evolutionary divergence (HED); wherein a high HED indicates a subject will be more responsive to immunotherapy.

In some embodiments an HED is determined by quantifying the sequence divergence between HLA class I alleles. In some embodiments an HED is determined by quantifying the sequence divergence between HLA class I alleles through measurement of the Grantham distance. In some embodiments an HED is determined as the mean evolutionary divergence of the HLA-A; HLA-B; and HLA-C genes.

In some embodiments, an immunotherapy is or comprises administration of one or more of PD-1 or PD-L1 blockade therapies. In some embodiments, an immunotherapy is or comprises administration of one or more of CTLA-4 blockade therapies. In some embodiments, an immunotherapy is or comprises administration of a combination of one or more of PD-1 blockade therapy and CTLA-4 blockade therapy. In some embodiments, an immunotherapy is or comprises administration of atezolizumab, avelumab, durvalumab, ipilimumab, nivolumab, pembrolizumab, or tremelimumab, and combinations therein.

### BRIEF DESCRIPTION OF THE DRAWING

FIGS. 1a to 1e demonstrate a landscape of HLA-I evolutionary divergences at HLA-A, B, and C. 1a, Schematic of experimental design. HLA-I evolutionary divergences (HED) are calculated between peptide-binding domains using the Grantham distance, a metric that incorporates physiochemical differences among amino acids, and used to stratify patients treated with immune checkpoint inhibitors. Predicted nonpeptides are called using whole-exome sequencing from the patient's tumor, counted, and correlated with genotype divergence. 1b, Hierarchical clustering of HED at HLA-A, HLA-B, and HLA-C(HLA-I). Heat map shows z-score normalized HED across all alleles across all patient cohorts used for downstream analyses. 1c, Comparison of HED at HLA-A, HLA-B, and HLA-C. ** P<0.01; *** P<0.001; Mann-Whitney test. 1d, Distribution of patient mean HED across all melanoma cohorts treated with ICI (ICI Melanoma) and TCGA (TCGA Melanoma). 1e, Distribution of patient mean HED across all melanoma cohorts treated with ICI (ICI Lung) and TCGA (TCGA Lung).

FIGS. 2a to 2e demonstrate that high mean HLA-I evolutionary divergence is associated with improved response and survival to immune checkpoint inhibitors. 2a, Association of high mean HLA-I evolutionary divergence (HED) (red) with improved survival after anti-CTLA-4 treatment in a cohort of metastatic melanoma patients fully heterozygous at HLA-I; P=0.0094; log-rank test. Density plots indicate the distribution and cutoff for mean HED used in the survival curves. T.Q.C.=top quartile cutoff, HR=hazard ratio, CI=confidence interval. 2b, Association of high mean HED (red) with improved survival after anti-PD-1 treatment in an independent cohort of patients with non-small cell lung cancer fully heterozygous at HLA-I; P=0.049; log-rank test. 2c, Association of high mean HED (red) with improved overall survival in an independent cohort of patients with melanoma fully heterozygous at HLA-I treated with anti-PD1; P=0.025; log-rank test. 2d, Association of high patient mean HED with clinical response (red) to ICI including all patients (both homozygous and heterozygous at HLA-I) for whom clinical response data were available from FIG. 2a-c); P=0.003; OR=0.35; Fisher's exact test. 2e, Association of high mean HED with clinical response to (red) ICI including only patients fully heterozygous at HLA-I for whom clinical response data were available from FIGS. 2a-c; P=0.03, OR=0.44; Fisher's exact test.

FIGS. 3a to 3h demonstrates the effect of mean HLA-I evolutionary divergence and tumor mutational burden on efficacy of immune checkpoint inhibitor treatment. 3a, Association of high mean HED (red) with improved overall survival after ICI in all patients (HLA-I homozygous or heterozygous) from FIG. 2 for whom tumor mutational burden (TMB) were available; P=0.0034; log-rank test. Density plot indicates the distribution and cutoff for mean HED used in the survival curves. T.Q.C.=top quartile cutoff, HR=hazard ratio, CI=confidence interval. 3b, Association of high TMB (red) with improved overall survival after ICI in patients from FIG. 3a; P=0.03; log-rank test. Density plot indicates the distribution and cutoff for TMB used in the survival curves. T.Q.C.=top quartile cutoff 3c, Survival of patients with both high mean HED and high TMB (red) after ICI treatment in patients from FIG. 3a; P=0.01; log-rank test 3d, Association of high mean HED (red) with improved overall survival after ICI in patients fully heterozygous at HLA-I from FIG. 2 for whom tumor mutational burden (TMB) were available; P=0.001; log-rank test. 3e, Association of high TMB with improved overall survival after ICI in patients from FIG. 3d; P=0.02; log-rank test. 3f, Survival of patients with both high mean HED and high TMB after ICI treatment in patients from FIG. 3a; P=0.007; log-rank test 3g, Cutpoint analysis showing the association of both high mean HED and high TMB with improved survival after ICI. Data show that the effect is present across all cutpoints (see Methods). 3h, Univariable Cox regression analysis showing the association of high HED (top quartile) at individual HLA-I loci with improved survival after ICI in patients from FIG. 3a (HLA-I homozygous or heterozygous, “all”) and FIG. 3d (fully heterozygous at HLA-I, “fully het”).

FIGS. 4a to 4f demonstrate that mean HLA-I evolutionary divergence is positively correlated with diversity of the tumor and human immunopeptidomes. 4a, Correlation of mean HED with number of unique neopeptides bound to alleles of each patient genotype using all patients fully heterozygous at HLA-I from FIG. 2 for whom neopeptide data were available; P=0.04; Kendall's rank correlation. Each point represents a patient HLA-I genotype (HLA-A, B, and C); y-axis depicts the mean number of neopeptides bound across HLA-A, B, and C (see Methods). 4b, Correlation of mean HED with TMB; P=0.46; Kendall's rank correlation. 4c, Correlation of mean HED with number of unique viral peptides bound to alleles of each HLA-I genotype; P<0.0001; Kendall's rank correlation. 4d, Correlation of mean HED with number of unique self peptides from the human proteome bound to alleles of each HLA-I genotype; P<0.001; Kendall's rank correlation. Y-axis depicts the mean number of self peptides bound across HLA-A, B, and C (see Methods). 4e, Association of mean HED with intratumoral TCR CDR3β clonality; P=0.02; Pearson's correlation. Red line indicates line of best linear fit. 4f, Schematic depicting the effects of HLA-I evolutionary divergence and TMB on immunopeptidome diversity and response to ICI. One representative HLA-I locus with high HED between alleles is depicted.

FIGS. 5a to 5c show hierarchical clustering of HLA-I evolutionary divergences at individual HLA class I loci. 5a, Hierarchical clustering of HED at HLA-A using all HLA-A alleles from all patient cohorts used for downstream analyses. 5b, Hierarchical clustering of HED at HLA-B using all HLA-B alleles. 5c, Hierarchical clustering of HED at HLA-C using all HLA-C alleles. Heat maps shows z-score normalized HED across all alleles. Red indicates high HED; blue indicates low HED.

FIGS. 6a to 6d demonstrate mean HLA-I evolutionary divergence is associated with improved response to immune checkpoint inhibitors. 6a, Association of high mean HED (red) with improved efficacy of anti-CTLA-4 treatment in a cohort of patients with metastatic melanoma; P=0.0072; log-rank test. Density plots indicate the distribution and cutoff used in the survival curves. T.Q.C.=top quartile cutoff, HR=hazard ratio, CI=confidence interval. 6b, Association of high (top quartile) tumor mutational burden (TMB) with overall survival after anti-CTLA-4 treatment in patients from FIG. 6a; P=0.20; log-rank test. 6c, Association of high mean HED and high TMB (red) with improved overall survival after anti-CTLA4 treatment in patients from FIG. 6a; P=0.024; log-rank test. 6d, Multivariable Cox proportional-hazards model including mean HED and other clinical variables. Data show independent effect of mean HED associated with improved survival after anti-CTLA-4. HED, TMB, and fraction of copy number alterations (FCNA) are dichotomized into high (1) and low (0) groups based on the top quartile for each variable.

FIGS. 7a to 7c demonstrate that neither HLA-I heterozygosity nor HLA-I evolutionary divergence is associated with prognosis in TCGA melanoma patients. 7a, Full heterozygosity at HLA-I (red) is not associated with prognosis in TCGA melanoma patients; P=0.14, log-rank test. 7b, High patient mean HED (red) is not associated with prognosis in patients from FIG. 7a; P=0.80, log-rank test. 7c, High mean HED (red) is not associated with prognosis in TCGA melanoma patients fully heterozygous at HLA-I; P=0.54; log-rank test.

FIGS. 8a to 8c shows neither HLA-I heterozygosity nor HLA-I evolutionary divergence is associated with prognosis in TCGA lung cancer patients. 8a, Full heterozygosity at HLA-I (red) is not associated with prognosis in TCGA lung cancer patients; P=0.38, log-rank test. 8b, High mean HED is not associated with prognosis in patients from FIG. 8a; P=0.73, log-rank test. 8c, High mean HED (red) is not associated with prognosis in TCGA lung cancer patients fully heterozygous at HLA-I; P=0.51, log-rank test.

FIGS. 9a to 9b shows the effects of mean HLA-I evolutionary divergence and tumor mutational burden are independent of cancer type and drug class. 9a, Multivariable Cox proportional-hazards model including mean HED and other clinical variables using patients from FIG. 3a (all patients, i.e. either HLA-I homozygous or heterozygous). Data show independent effect of mean HED in predicting response to ICI. 9b, Multivariable Cox proportional-hazards model including mean HED and other clinical variables using patients from FIG. 3d (patients fully heterozygous at HLA-I). Data show independent effect of mean HED associated with improved survival after ICI therapy. HED and TMB are dichotomized into high (1) and low (0) groups using the top quartile for each variable.

FIG. 10 provides an oncoprint showing mutations in genes in the patient cohorts. Data show no difference in proportion of patients with mutations in the presented genes between patients with high mean HLA-I evolutionary divergence (HED) and low mean HED. LOH=loss of heterozygosity ay HLA-I.

FIGS. 11a to 11i demonstrate the association of HLA-I evolutionary divergence at each class I locus with diversity of tumor and human immunopeptidomes. 11a, Correlation of HED at HLA-A with number of unique neopeptides bound to HLA-A alleles of each patient genotype using all patients heterozygous at HLA-A from FIG. 2 for whom neopeptide data were available; P=0.15. Each point represents a patient HLA-A genotype 11b, Correlation of HED at HLA-B with number of unique neopeptides bound to HLA-B alleles of each patient genotype using patients heterozygous at HLA-B; P=0.001 11c, Correlation of HED at HLA-C with number of unique neopeptides bound to HLA-C alleles of each patient genotype using patients heterozygous at HLC-C; P=0.03. d, Correlation of HED at HLA-A with number of unique viral peptides bound to HLA-A alleles of each patient genotype using patients heterozygous at HLA-A; P=0.01. e, Correlation of HED at HLA-B with number of unique viral peptides bound to HLA-B alleles of each patient genotype using patients heterozygous at HLA-B; P<0.0001. f, Correlation of HED at HLA-C with number of unique viral peptides bound to HLA-C alleles of each patient genotype using patients heterozygous at HLA-C; P<0.0001. g, Correlation of HED at HLA-A with number of unique self peptides bound to HLA-A alleles of each patient genotype using patients heterozygous at HLA-A; P=0.79. h, Correlation of HED at HLA-B with number of unique self peptides bound to HLA-B alleles of each patient genotype using patients heterozygous at HLA-B; P<0.0001. i, Correlation of HED at HLA-C with number of unique self peptides bound to HLA-C alleles of each patient genotype using patients heterozygous at HLA-C; P=0.79. All p-values were calculated using Kendall's rank correlation. Red line indicates line of best linear fit.

FIGS. 12a to 12b demonstrate association of HLA-I evolutionary divergence at HLA-A and HLA-B with diversity of the self immunopeptidome generated by mass spectrometry. 12a, Correlation of HED at HLA-A with number of unique naturally processed self peptides bound to alleles of each HLA-A genotype from patients with metastatic melanoma patients heterozygous at each HLA-I from Pearson et al; P=0.46; Data recapitulate results derived from computational peptide-HLA binding predictions shown in FIG. 11d. 12b, Correlation of HED at HLA-B with number of unique naturally processed self peptides bound to alleles of each HLA-B genotype; P=0.03. Data recapitulate results derived from computational peptide-HLA binding predictions shown in FIG. 11e. All p-values were calculated using Kendall's rank correlation. Red line indicates line of best fit.

FIG. 13 demonstrates the effect of mean HLA-I evolutionary divergence on hazard ratio from survival across all possible cutpoints. Cutpoint analysis showing the relationship between mean HED and hazard ratio. Data show a negative relationship between mean HED and hazard ratio across all possible cutpoints for mean HED, indicating improved overall survival as mean HED increases.

FIGS. 14a-14b shows the combined effect of HED and TMB on survival after ICI administration and multivariable analysis of HED at individual loci. a, Cutpoint analysis showing the association of both high mean HED and high TMB with improved survival after ICI (same distributions as FIG. 3g). Data show a reduction in hazard ratio when combining HED and TMB compared to either variable alone. Green indicates log-rank p-value<0.05; red indicates non-significant log-rank p-value. b, Multivariable cox regression analysis demonstrating the effect of HED at individual loci on overall survival after ICI administration. Data indicate that high HED at HLA-A and HLA-B are each associated with improved overall survival after ICI.

FIGS. 15a-15e show the effect of mean HLA-I evolutionary divergence and tumor mutational burden on efficacy of immune checkpoint inhibitor treatment in an independent set of patients 15a, Association of high mean HED (red) with improved overall survival after ICI in an independent pan-cancer dataset of patients described in Chowell et al. These patients do not overlap with those presented in FIGS. 2 & 3. Cutoff was determined using the median mean HED across the cohort. P=0.02; log-rank test. HR=hazard ratio, CI=confidence interval. 15b, Association of high mean HED (red) with improved overall survival after ICI in an independent, pan-cancer dataset of patients described in Chowell et al. These patients do not overlap with those presented in FIGS. 2 & 3. Patients in the red curve have mean HED greater than or equal to the top quartile; patients in the blue curve have mean HED less than or equal to the first quartile. P=0.04; log-rank test 15c, Association of high mean HED (red) with improved overall survival after ICI in all patients described in Chowell et al. Cutoff was determined using the median mean HED across the cohort. P=0.0012; log-rank test. 15d, Association of high mean HED (red) with improved overall survival after ICI in all patients described in Chowell et al. Patients in the red curve have mean HED greater than or equal to the top quartile; patients in the blue curve have mean HED less than or equal to the first quartile. P=0.0037; log-rank test. 15e, Multivariable Cox proportional-hazards model including mean HED and other variables. Data show independent effect of mean HED on improved survival after ICI administration when adjusting for TMB and cancer type. Mean HED is dichotomized into high (1) and low (0) groups using the median; TMB is treated as a continuous variable. f, Association of mean HED and TMB with overall survival after ICI in all patients from Chowell et al for whom TMB were available. Patients were first stratified by TMB using the top quartile, and then stratified by mean HED using the top quartile within each of the high TMB and low TMB groups. P-value calculated using the log-rank test.

FIGS. 16a-16e demonstrates validation of Grantham distance score between alleles using the mass spectrometry peptidomes derived from mono-allelic cells by Abelin et al. 16a, The Grantham distance score, used here to estimate HLA evolutionary divergence (HED) between HLA alleles, correlates negatively with the overlap of peptides bound by any two HLA alleles from the dataset of Abelin et al., which contains naturally eluted peptide repertoires from mono-allelic cell lines of 16 different HLA-I alleles (representing 120 possible allele pairs). 16b, Same as a, for HLA-A alleles alone. 16c, Same as a, for HLA-B alleles alone. All p-values calculated were calculated using Kendall's rank correlation. Blue line indicates line of best fit. 16d, HED at HLA-A is positively correlated with the total number of peptides bound to each pair of HLA-A alleles. 16e, Same as d, for HLA-B alleles.

FIG. 17 demonstrates evolutionary HLA class I divergence (HED) predicts response to combination treatment with lenvatinib (anti-VEGFR) plus pembrolizumab (anti-PD-1) in patients with renal cell carcinoma.

### DEFINITIONS

In order for the present invention to be more readily understood, certain terms are defined below. Those skilled in the art will appreciate that definitions for certain terms may be provided elsewhere in the specification, and/or will be clear from context.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody agent: As used herein, the term “antibody agent” refers to an agent that specifically binds to a particular antigen. In some embodiments, the term encompasses any polypeptide with immunoglobulin structural elements sufficient to confer specific binding. Suitable antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular Immuno Pharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody polypeptide: As used herein, the terms “antibody polypeptide” or “antibody”, or “antigen-binding fragment thereof”, which may be used interchangeably, refer to polypeptide(s) capable of binding to an epitope. In some embodiments, an antibody polypeptide is a full-length antibody, and in some embodiments, is less than full length but includes at least one binding site (comprising at least one, and preferably at least two sequences with structure of antibody “variable regions”). In some embodiments, the term “antibody polypeptide” encompasses any protein having a binding domain which is homologous or largely homologous to an immunoglobulin-binding domain. In particular embodiments, “antibody polypeptides” encompasses polypeptides having a binding domain that shows at least 99% identity with an immunoglobulin binding domain. In some embodiments, “antibody polypeptide” is any protein having a binding domain that shows at least 70%, 80%, 85%, 90%, or 95% identity with an immuoglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source or antibody library, recombinant production in or with a host system, chemical synthesis, etc., or combinations thereof. An antibody polypeptide may be monoclonal or polyclonal. An antibody polypeptide may be a member of any immunoglobulin class, including any of the human classes: IgG, IgM, IgA, IgD, and IgE. In certain embodiments, an antibody may be a member of the IgG immunoglobulin class. As used herein, the terms “antibody polypeptide” or “characteristic portion of an antibody” are used interchangeably and refer to any derivative of an antibody that possesses the ability to bind to an epitope of interest. In certain embodiments, the “antibody polypeptide” is an antibody fragment that retains at least a significant portion of the full-length antibody's specific binding ability. Examples of antibody fragments include, but are not limited to, Fab, Fab′, F(ab′)₂, scFv, Fv, dsFv diabody, and Fd fragments. Alternatively or additionally, an antibody fragment may comprise multiple chains that are linked together, for example, by disulfide linkages. In some embodiments, an antibody polypeptide may be a human antibody. In some embodiments, the antibody polypeptides may be a humanized. Humanized antibody polypeptides include may be chimeric immunoglobulins, immunoglobulin chains or antibody polypeptides (such as Fv, Fab, Fab′, F(ab′)2 or other antigen-binding subsequences of antibodies) that contain minimal sequence derived from non-human immunoglobulin. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In particular embodiments, antibody polypeptides for use in accordance with the present invention bind to particular epitopes of on immune checkpoint molecules.

Antigen: An “antigen” is a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Blockade”: The term “blockade” as used herein, refers to an entity or event whose presence or level correlates with a reduction in level and/or activity of an indicated target. Thus, for example, “a PD-1 blockade” is an agent or event whose presence correlates with reduction in level and/or activity of PD-1. In some such embodiments, a relevant activity of PD-1 may be or comprise interaction with one of more of its ligands (e.g., PD-L1 and/or PD-L2) and/or a downstream effect thereof. In some embodiments, a PD-1 blockade may be achieved by administration of an agent, such as an antibody agent, that targets PD-1 and/or a PD-1 ligand (e.g., PD-L1 and/or PD-L2) and/or a complex thereof. In some particular embodiments, a PD-1 blockade may be achieved through administration of an antibody agent that binds to PD-1. In some embodiments, a PD-1 blockade may be achieved through administration of one or more of nivolumab, pembrolizumab, atezolizumab, avelumab, and/or durvalumab. Analogously, a “CTL4-blockade is an agent or event whose presence correlates with reduction in level and/or activity of CTLA-4. In some such embodiments, a relevant activity of CTLA-4 may be or comprise interaction with one of more of its ligands (e.g., CD80 and/or CD86) and/or a downstream effect thereof. In some embodiments, a CTLA-4 blockade may be achieved by administration of an agent, such as an antibody agent, that targets CTLA-4 ligand (e.g., CD80 and/or CD86) and/or a complex thereof. In some particular embodiments, a CTLA-4 blockade may be achieved through administration of an antibody agent that binds to CTLA-4. In some embodiments, a CTLA-4 blockade may be achieved through administration of one or more of ipilimumab and/or tremelimumab.

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Comparable: The term “comparable” is used herein to describe two (or more) sets of conditions, circumstances, individuals, or populations that are sufficiently similar to one another to permit comparison of results obtained or phenomena observed. In some embodiments, comparable sets of conditions, circumstances, individuals, or populations are characterized by a plurality of substantially identical features and one or a small number of varied features. Those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those skilled in the art will appreciate that relative language used herein (e.g., enhanced, activated, reduced, inhibited, etc.) will typically refer to comparisons made under comparable conditions.

Consensus sequence: As used herein, the term “consensus sequence” refers to a core sequence that elicits or drives a physiological phenomenon (e.g., an immune response). It is to be understood by those of skill in the art that a a cancer cell that shares a “consensus sequence” with an antigen of an infectious agent shares a portion of amino acid sequence that affects the binding affinity of the antigen to an MHC molecule (either directly or allosterically), and/or facilitates recognition by T cell receptors. In some embodiments, a consensus sequence is a tetrapeptide. In some embodiments, a consensus sequence is a nonapeptide. In some embodiments, a consensus sequence is between four and nine amino acids in length. In some embodiments, a consensus sequence is greater than nine amino acids in length.

Diagnostic information: As used herein, diagnostic information or information for use in diagnosis is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome, when administered across a population of patients.

Durable clinical benefit: As used herein, the term “durable clinical benefit” (DCB), has its art-understood meaning, referring to a clinical benefit that lasts for a relevant period of time. In some embodiments, such a clinical benefit is or comprises reduction in tumor size, increase in progression free survival, increase in overall survival, decrease in overall tumor burden, decrease in the symptoms caused by tumor growth such as pain, organ failure, bleeding, damage to the skeletal system, and other related sequelae of metastatic cancer and combinations thereof. In some embodiments, the relevant period of time is at least 1 month, 2 months, 3 months, 4 months, 5 months, 6 months, 7 months, 8 months, 9 months, 10 months, 11 months, 1 year, 2 years, 3 years, 4 years, 5 years, or longer. In some particular embodiments, the relevant period of time is 6 months.

Exome: As used herein, the term “exome” is used in accordance with its art-understood meaning referring to the set of exon sequences that are found in a particular genome.

Favorable response: As used herein, the term “favorable response” refers to a reduction in frequency and/or intensity of one or more symptoms, reduction in tumor burden, full or partial remission, or other improvement in disease pathophysiology. Symptoms are reduced when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many cancer patients with smaller tumors have no symptoms. It is not intended that the present invention be limited only to cases where the symptoms are eliminated. The present invention specifically contemplates treatment such that one or more symptoms is/are reduced (and the condition of the subject is thereby “improved”), albeit not completely eliminated. In some embodiments, a favorable response is established when a particular therapeutic regimen shows a statistically significant effect when administered across a relevant population; demonstration of a particular result in a specific individual may not be required. Thus, in some embodiments, a particular therapeutic regimen is determined to have a favorable response when its administration is correlated with a relevant desired effect.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Immune checkpoint modulator: As used herein, the term “immune checkpoint modulator” refers to an agent that interacts directly or indirectly with an immune checkpoint. In some embodiments, an immune checkpoint modulator increases an immune effector response (e.g., cytotoxic T cell response), for example by stimulating a positive signal for T cell activation. In some embodiments, an immune checkpoint modulator increases an immune effector response (e.g., cytotoxic T cell response), for example by inhibiting a negative signal for T cell activation (e.g. disinhibition). In some embodiments, an immune checkpoint modulator interferes with a signal for T cell anergy. In some embodiments, an immune checkpoint modulator reduces, removes, or prevents immune tolerance to one or more antigens.

Long Term Benefit: In general, the term “long term benefit” refers to a desirable clinical outcome, e.g., observed after administration of a particular treatment or therapy of interest, that is maintained for a clinically relevant period of time. To give but one example, in some embodiments, a long term benefit of cancer therapy is or comprises (1) no evidence of disease (“NED”, for example upon radiographic assessment) and/or (2) stable or decreased volume of diseases. In some embodiments, a clinically relevant period of time is at least 1 month, at least 2 months, at least 3 months, at least 4 months, at least 5 months or more. In some embodiments, a clinically relevant period of time is at least six months. In some embodiments, a clinically relevant period of time is at least 1 year.

Marker: A marker, as used herein, refers to an agent whose presence or level is a characteristic of a particular tumor or metastatic disease thereof. For example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Modulator: The term “modulator” is used to refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Mutation: As used herein, the term “mutation” refers to permanent change in the DNA sequence that makes up a gene. In some embodiments, mutations range in size from a single DNA building block (DNA base) to a large segment of a chromosome. In some embodiments, mutations can include missense mutations, frameshift mutations, duplications, insertions, nonsense mutation, deletions and repeat expansions. In some embodiments, a missense mutation is a change in one DNA base pair that results in the substitution of one amino acid for another in the protein made by a gene. In some embodiments, a nonsense mutation is also a change in one DNA base pair. Instead of substituting one amino acid for another, however, the altered DNA sequence prematurely signals the cell to stop building a protein. In some embodiments, an insertion changes the number of DNA bases in a gene by adding a piece of DNA. In some embodiments, a deletion changes the number of DNA bases by removing a piece of DNA. In some embodiments, small deletions may remove one or a few base pairs within a gene, while larger deletions can remove an entire gene or several neighboring genes. In some embodiments, a duplication consists of a piece of DNA that is abnormally copied one or more times. In some embodiments, frameshift mutations occur when the addition or loss of DNA bases changes a gene's reading frame. A reading frame consists of groups of 3 bases that each code for one amino acid. In some embodiments, a frameshift mutation shifts the grouping of these bases and changes the code for amino acids. In some embodiments, insertions, deletions, and duplications can all be frameshift mutations. In some embodiments, a repeat expansion is another type of mutation. In some embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In some embodiments, a repeat expansion is a mutation that increases the number of times that the short DNA sequence is repeated.

“Mutational Load”: The term “mutational load” is used herein to refer to the number of mutations detected in a sample (e.g., a tumor sample) at a given point in time. Those skilled in the art will appreciate that “mutational load” may also be referred to as “mutational burden”. In some embodiments, mutations included in an assessment of mutational load may be neoantigen mutations (i.e., mutations that give rise to neoantigens). In some embodiments, a sample in which mutational load is assessed is from a single tumor. In some embodiments, a sample is pooled from multiple tumors, either from a single individual subject, or from a plurality of subjects.

Neoepitope: A “neoepitope” is understood in the art to refer to an epitope that emerges or develops in a subject after exposure to or occurrence of a particular event (e.g., development or progression of a particular disease, disorder or condition, e.g., infection, cancer, stage of cancer, etc.). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neopepitope is one that triggers an immune response that kills or otherwise destroys cells that express it (e.g., at a relevant level). In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope). In some embodiments, a neoepitope is a neoantigen.

No Benefit: As used herein, the phrase “no benefit” is used to refer to absence of detectable clinical benefit (e.g., in response to administration of a particular therapy or treatment of interest). In some embodiments, absence of clinical benefit refers to absence of statistically significant change in any particular symptom or characteristic of a particular disease, disorder, or condition. In some embodiments, absence of clinical benefit refers to a change in one or more symptoms or characteristics of a disease, disorder, or condition, that lasts for only a short period of time such as, for example, less than about 6 months, less than about 5 months, less than about 4 months, less than about 3 months, less than about 2 months, less than about 1 month, or less. In some embodiments, no benefit refers to no durable benefit.

Objective Response: As used herein, the phrase “objective response” refers to size reduction of a cancerous mass by a defined amount. In some embodiments, the cancerous mass is a tumor. In some embodiments, confirmed objective response is response confirmed at least four (4) weeks after treatment.

Objective Response Rate: As used herein, the term “objective response rate” (“ORR”) has its art-understood meaning referring to the proportion of patients with tumor size reduction of a predefined amount and for a minimum time period. In some embodiments, response duration usually measured from the time of initial response until documented tumor progression. In some embodiments, ORR involves the sum of partial responses plus complete responses.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the disorder or condition is metastatic cancer.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Prognostic and predictive information: As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Progression Free Survival: As used herein, the term “progression free survival” (PFS) has its art-understood meaning relating to the length of time during and after the treatment of a disease, such as cancer, that a patient lives with the disease but it does not get worse. In some embodiments, measuring the progression-free survival is utilized as an assessment of how well a new treatment works. In some embodiments, PFS is determined in a randomized clinical trial; in some such embodiments, PFS refers to time from randomization until objective tumor progression and/or death.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Reference: Those of skill in the art will appreciate that, in many embodiments described herein, a determined value or characteristic of interest is compared with an appropriate reference. In some embodiments, a reference value or characteristic is one determined for a comparable cohort, individual, population, or sample. In some embodiments, a reference value or characteristic is tested and/or determined substantially simultaneously with the testing or determination of the characteristic or value of interest. In some embodiments, a reference characteristic or value is or comprises a historical reference, optionally embodied in a tangible medium. Typically, as would be understood by those skilled in the art, a reference value or characteristic is determined under conditions comparable to those utilized to determine or analyze the characteristic or value of interest.

Response: As used herein, the term “response” may refer to an alteration in a subject's condition that occurs as a result of or correlates with treatment. In some embodiments, a response is or comprises a beneficial response. In some embodiments, a beneficial response may include stabilization of the condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In some embodiments, “response” may refer to response of an organism, an organ, a tissue, a cell, or a cell component or in vitro system. In some embodiments, a response is or comprises a clinical response. In some embodiments, presence, extent, and/or nature of response may be measured and/or characterized according to particular criteria; in some embodiments, such criteria may include clinical criteria and/or objective criteria. In some embodiments, techniques for assessing response may include, but are not limited to, clinical examination, positron emission tomography, chest X-ray CT scan, MRI, ultrasound, endoscopy, laparoscopy, presence or level of a particular marker in a sample, cytology, and/or histology. Where a response of interest is or comprises response of a tumor to therapy, those of ordinary skill will be aware of a variety of established techniques for assessing such response, including, for example, for determining tumor burden, tumor size, tumor stage, etc. For example, certain technologies for assessing response of solid tumors to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. Those of ordinary skill in the art will be aware of, and/or will appreciate in light of the present disclosure, strategies for determining particular response criteria for individual tumors, tumor types, patient populations or cohorts, etc., as well as for determining appropriate references therefor.

Sample: As used herein, the term “sample” typically refers to a biological sample obtained or derived from a source of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchioalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specific binding: As used herein, the terms “specific binding” or “specific for” or “specific to” refer to an interaction (typically non-covalent) between a target entity (e.g., a target protein or polypeptide) and a binding agent (e.g., an antibody, such as a provided antibody). As will be understood by those of ordinary skill, an interaction is considered to be “specific” if it is favored in the presence of alternative interactions. In many embodiments, an interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. In particular embodiments, an antibody specific for receptor tyrosine kinases has less than 10% cross-reactivity with receptor tyrosine kinase bound to protease inhibitors (e.g., ACT). One of ordinary skill in the art will be able to select antibodies having a sufficient degree of specificity to perform appropriately in any given application (e.g., for detection of a target molecule, for therapeutic purposes, etc.). Specificity may be evaluated in the context of additional factors such as the affinity of the binding molecule for the target molecule versus the affinity of the binding molecule for other targets (e.g., competitors).

Stage of cancer: As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

Subject: As used herein, the term “subject” or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition (e.g., a cancer) has been diagnosed with and/or exhibits one or more symptoms of the disease, disorder, or condition. In some embodiments, an individual who is suffering from cancer has cancer, but does not display any symptoms of cancer and/or has not been diagnosed with a cancer.

Susceptible to: An individual who is “susceptible to” a disease, disorder, or condition (e.g., cancer) is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who displays conditions associated with development of the disease, disorder, or condition. In some embodiments, a risk of developing a disease, disorder, and/or condition is a population-based risk.

Target cell or target tissue: As used herein, the terms “target cell” or “target tissue” refer to any cell, tissue, or organism that is affected by a condition described herein and to be treated, or any cell, tissue, or organism in which a protein involved in a condition described herein is expressed. In some embodiments, target cells, target tissues, or target organisms include those cells, tissues, or organisms in which there is a detectable amount of immune checkpoint signaling and/or activity. In some embodiments, target cells, target tissues, or target organisms include those cells, tissues or organisms that display a disease-associated pathology, symptom, or feature.

Therapeutic regimen: As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include a treatment or series of treatments designed to achieve a particular effect, e.g., reduction or elimination of a detrimental condition or disease such as cancer. The treatment may include administration of one or more compounds either simultaneously, sequentially or at different times, for the same or different amounts of time. Alternatively, or additionally, the treatment may include exposure to radiation, chemotherapeutic agents, hormone therapy, or surgery. In addition, a “treatment regimen” may include genetic methods such as gene therapy, gene ablation or other methods known to reduce expression of a particular gene or translation of a gene-derived mRNA.

Therapeutic agent: As used herein, the phrase “therapeutic agent” refers to any agent that, when administered to a subject, has a therapeutic effect and/or elicits a desired biological and/or pharmacological effect.

Therapeutically effective amount: As used herein, the term “therapeutically effective amount” refers to an amount of an agent (e.g., an immune checkpoint modulator) that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a substance (e.g., provided compositions) that partially or completely alleviates, ameliorates, relieves, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

### DETAILED DESCRIPTION OF CERTAIN EMBODIMENTS

### Human Leukocyte Antigen

One factor that has been repeatedly associated with modulating the immune response during bacterial or viral infection, inflammatory conditions, and autoimmune diseases, is HLA class I genotype. The human leukocyte antigen (HLA) complex is a gene complex encoding the major histocompatibility complex (MHC) proteins. The major histocompatibility complex (MHC) class I coding region in humans is central to the immune response. Each HLA class I molecule binds specific peptides derived from intracellular proteins that have been processed and transported into the endoplasmic reticulum by the TAP proteins, where they are bound to the MHC class I molecules for presentation on the cell surface.

The primary function of the major histocompatibility complex class I (MHC-I) molecules, which are encoded by HLA-I, is the presentation of self and non-self peptides for recognition by cytotoxic T-cells^{20,21}. Each individual's HLA-I genotype consists of a pair of alleles at each of the classical class I genes—HLA-A, B, and C, and their polymorphism is concentrated within their peptide-binding domains²¹⁻²⁴. The set of peptides bound by each MHC-I molecule is collectively referred to as its immunopeptidome, and different HLA-I alleles have different peptide-binding specificities with varying degrees of overlap according to the amount of physiochemical sequence divergence between them^{20,25-29}. The concomitant diversity of HLA-I genotypes and peptide-binding specificities yields marked variability in the diversity of peptide repertoires that can be displayed by the MHC-I complex across individuals^{20,29}. Accordingly, this variation may affect the ability of each individual's immune system to recognize tumor antigens and consequently may influence response to ICI.

MHC molecules are extremely polymorphic, and over a thousand allelic variants have already been described at the class I A and B loci. Most of the polymorphism is located in the peptide-binding region, and as a result each variant is believed to bind a unique repertoire of peptide ligands. Despite this polymorphism, HLA class I molecules can be clustered into groups, designated as supertypes (a.k.a. superfamilies), representing sets of molecules that share largely overlapping peptide binding specificity. Exemplary supertypes include but are not limited to A02, A24, A03, B07, B27, B44, Each supertype can be described by a supermotif that reflects the broad main anchor motif recognized by molecules within the corresponding supertype. For example, molecules of the A02-supertype share specificity for peptides with aliphatic hydrophobic residues in position 2 and at the C-terminus, while A03-supertype molecules recognize peptides with small or aliphatic residues in position 2 and basic residues at the C-terminus.

Typically, in the case of human leukocyte antigen (HLA) class I, the main binding energy is provided by the interaction of residues in position 2 and the C-terminus of the peptide with the B and F binding pockets of the MHC molecule, respectively although side chains throughout the ligand can have a positive or negative influence on binding capacity. Once pathogen or tumor-derived epitopes are presented on the cell surface, CD8+ T-cells must be able to recognize them to subsequently elicit an immune response and eliminate cells bearing those same epitopes. Some tumor cells have reduced ability to present epitopes on the surface due to genetic alterations resulting in loss of heterozygosity (LOH) in the HLA locus. LOH is a gross chromosomal event that results in loss of an entire gene and surrounding chromosomal region. The anti-tumor activity of immune checkpoint treatment has been shown to depend on CD8+ T cell, MHC class I-dependent immune activity.

Among other things, the present disclosure demonstrates that HLA class I genotype can influence clinical efficacy of immunotherapy treatment for cancer. The present disclosure establishes, among other things, that the sequence dissimilarity or divergence between the two alleles at one or more of the HLA class I loci (e.g., A, B, or C), here also called HLA class I evolutionary divergence (HED), can influence clinical efficacy of immunotherapy treatment.

The present disclosure establishes, among other things, that the calculation of the HED of an individual is independent of the technique or protocol used to genotype the individual or patient at their HLA genes. HED is calculated based on an individual's sequence variants of the HLA genes (among others HLA-A, HLA-B, HLA-C), either directly from the amino acid sequences or after translation from the nucleotide sequences. In some embodiments, HLA genotype information and HLA allele sequences will be characterized following the established standard HLA allele nomenclature as described on the website http://hla.alleles.org/. Those of ordinary skill in the art will appreciate that HLA genotypes and HLA allele sequences can be determined through various techniques, including sequence-specific oligo probes, sequence-specific amplification and sequencing through either Sanger or Next Generation sequencing technology, imputation from other genomic information such as from SNP marker genotypes, target-capture approaches, or read mapping from shotgun sequence data.

In some embodiments, an individual's HED value is considered high if it is higher than the average HED of a population of individuals. In some embodiments, an individual's HED value is considered high if it is higher than the average HED of a specific study cohort of individuals, such as a cohort of healthy individuals or a cohort of individuals with poor treatment response. In some embodiments, an individual's HED value is considered high if it is higher than the average HED value of a group of HLA-homozygous individuals. Those of ordinary skill in the art will appreciate that the decision of whether an individual's HED value is considered high in certain cases can depend on the relative distribution of HED values in the study population.

The present disclosure establishes, among other things, that in certain cases, individuals with high mean HED at one or more HLA class I loci (e.g., A, B, or C) are more likely to respond positively to immunotherapy. In some embodiments, individuals with high mean HED at all three HLA class I loci are more likely to respond positively to immunotherapy.

Further, the present disclosure establishes, among other things, that in certain cases, individuals with high mean HED at one or more HLA class I loci (e.g., A, B, or C) and higher tumor mutational loads as described herein are more likely to respond positively to immunotherapy than individuals with lower tumor mutational loads and/or no or low mean HED. In some embodiments, individuals with high mean HED at all three HLA class I loci and higher tumor mutational loads as described herein are more likely to respond positively to immunotherapy than individuals with significantly lower tumor mutational loads and/or low mean HED at HLA class I loci.

In some embodiments, a subject's HED can be determined by measuring Grantham distance, which appears to be most appropriate, or any other metric that quantifies the difference in sequence between the two alleles at a given HLA locus, particularly along the exons of the peptide-binding domains. Such metrics include, among others, the p-distance (proportional difference in residues between alleles) or distance measures based on the Dayhoff, JTT or Sandberg dissimilarity matrices (Pierini & Lenz 2018 Mol Biol Evol³²). Using one of these or similar metrics, the HED can be characterized in different ways, for instance by using the mean or median HED across several or all studied HLA loci or an appropriate reference population of HLA loci, by using the cumulative HED across several or all studied HLA loci or an appropriate reference population of HLA loci, or by using the HED between the two alleles of one HLA locus only (e.g. HLA-A, HLA-B, or HLA-C). Those of ordinary skill in the art will appreciate that these are largely equivalent approaches to estimating the HED of an individual. In some embodiments, an appropriate reference can be a population of individuals. In some embodiments, an appropriate reference can be a cohort of individuals. In some embodiments, an appropriate reference can be a population or cohort of healthy individuals. In some embodiments, an appropriate reference can be a population or cohort of individuals diagnosed with cancer. In some embodiments, an appropriate reference can be a population or cohort of individuals with poor treatment response. In some embodiments, an appropriate reference can be a population of HLA-homozygous individuals.

### Immunotherapy

In some embodiments, the present disclosure relates to administration of immunotherapy to a subject. In some embodiments, immunotherapy is or comprises immune checkpoint modulation therapy. In some embodiments, immunotherapy involves administration of one or more immunomodulatory agents; in some embodiments an immunomodulatory agent is or comprises an immune checkpoint modulator. In some embodiments, an immune checkpoint modulator is an agent (e.g., an antibody agent) that targets (i.e., specifically interacts with) an immune checkpoint target. In some embodiments, an immune checkpoint target is or comprises one or more of CTLA-4, PD-1, PD-L1, GITR, OX40, LAG-3, KIR, TIM-3, CD28, CD40, and CD137; in some embodiments, immune checkpoint modulator therapy is or comprises administration of an antibody agent that targets one or more such immune checkpoint targets.

Alternatively or additionally, in some embodiments, the present disclosure provides technologies for assessing immunotherapy (e.g., immune checkpoint modulator therapy), including for assessing effectiveness of one or more particular therapeutic regimen(s) for treatment of particular subjects or subject populations and/or of particular cancers or tumors. For example, in some embodiments, the present disclosure provides technologies for assessing particular therapies or regimens for effectiveness in treatment in light of an HED level. In some embodiments, such assessment may involve administering the therapy or regimen to a population of individuals, tissues (e.g., tumors or samples thereof), or cells with different HED levels and determining its effectiveness therein. Alternatively or additionally, in some embodiments, such assessment may involve administering the therapy or regimen to a subject(s), tissue(s) (e.g., tumor or sample thereof), or cell(s) with a particular HED and determining its effectiveness, optionally relative to an appropriate control (e.g., a positive and/or a negative control) treatment. In some embodiments, a therapy or regimen assessed as described herein is administered to a subject or population of subjects having an HED with respect to which the therapy or regimen has been determined to be effective.

In some embodiments, immune checkpoint refers to inhibitory pathways of an immune system that are responsible for maintaining self-tolerance and modulating duration and amplitude of physiological immune responses. Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, among other things, immune checkpoint modulators may be administered to overcome inhibitory signals and permit and/or augment an immune attack against cancer cells. Immune checkpoint modulators may facilitate immune cell responses against cancer cells by decreasing, inhibiting, or abrogating signaling by negative immune response regulators (e.g. CTLA-4), or may stimulate or enhance signaling of positive regulators of immune response (e.g. CD28).

Advances in understanding of molecular mechanisms of T cell activation and inhibition and immune homeostasis have allowed for rational development of immunologically targeted therapies for cancer. The best known of these are immune checkpoint modulator monoclonal antibodies that block CTLA-4 and PD-1 pathways, representing critical inhibitory checkpoints that restrain T cells from full and persistent activation and proliferation under normal physiologic conditions. Blockade of CTLA-4 and/or PD-1 pathways can result in durable regressions for patients with a widening spectrum of malignancies. In some embodiments, an immunotherapy is or comprises administration of one or more of PD-1 or PD-L1 blockade therapies. In some embodiments, an immunotherapy is or comprises administration of one or more of CTLA-4 blockade therapies. In some embodiments, an immunotherapy can include any of ipilumimab and tremelimumab which target CTLA-4; pembrolizumab, nivolumab, avelumab, durvalumab, and atezoluzumab, which target PD-1; or combinations thereof.

Teachings of the present disclosure, among other things, predict responsiveness to immune checkpoint modulators, and particularly to therapeutic modalities or regimens targeting immune checkpoint regulators. The present disclosure, among other things, demonstrates that HED correlates with responsiveness to immune checkpoint modulators. In some embodiments, the present disclosure demonstrates that high mean HED correlates with an increased likelihood of clinical efficacy from immune checkpoint regulators for those cancers responsive to immunotherapy (e.g., to PD-1 blockade and/or to CTLA-4 blockade). In some embodiments, the present disclosure demonstrates that high cumulative HED correlates with an increased likelihood of clinical efficacy from immune checkpoint regulators for those cancers responsive to immunotherapy. In some embodiments, immunotherapy (e.g. immune checkpoint modulator therapy) involves administration of an agent that acts as a blockade of cytotoxic T-lymphocyte-associated protein 4 (CTLA-4). In certain embodiments, immunotherapy involves treatment with an agent that interferes with an interaction involving CTLA-4 (e.g., with CD80 or CD86). In some embodiments, immunotherapy involves administration of one or more of tremelimumab and/or ipilimumab. In some embodiments, immunotherapy (e.g. immune checkpoint modulator therapy) involves administration of an agent (e.g. antibody agent) that acts as a blockade of programmed cell death 1 (PD-1). In certain embodiments, immunotherapy involves treatment with an agent that interferes with an interaction involving PD-1 (e.g., with PD-L1). In some embodiments, immunotherapy involves administration of an agent (e.g. antibody agent) that specifically interacts with PD-1 or with PD-L1. In some embodiments, immunotherapy (e.g. immune checkpoint modulator therapy) involves administration of one or more of nivolumab, pembrolizumab, atezolizumab, avelumab, and/or durvalumab.

### CTLA-4

CTLA-4 is a member of the immunoglobulin superfamily that is expressed by activated T cells and transmits an inhibitory signal to T cells. CTLA-4 is structurally similar to T cell co-stimulatory protein, CD28, and both molecules bind to CD80 and CD86 on antigen-presenting cells.¹⁸ CTLA-4 binds CD80 and CD86 with greater affinity than CD28, thus enabling it to outcompete CD28 for its ligands.¹⁸ CTLA-4 transmits an inhibitory signal to T cells, whereas CD28 transmits a stimulatory signal. T cell activation through T cell receptor and CD28 leads to increased expression of CTLA-4.

The mechanism by which CTLA-4 acts in T cells remains somewhat elusive. Biochemical evidence suggests that CTLA-4 recruits a phosphatase to a T cell receptor, thus attenuating the signal. It has also been suggested that CTLA-4 may function in vivo by capturing and removing CD80 and CD86 from membranes of antigen-presenting cells, thus making these antigens unavailable for triggering of CD28.

CTLA-4 protein contains an extracellular V domain, a transmembrane domain, and a cytoplasmic tail. CTLA-4 has an intracellular domain that is similar to that of CD28, in that it has no intrinsic catalytic activity and contains one YVKM motif able to bind PI3K, PP2A and SHP-2, as well as one proline-rich motif able to bind SH3-containing proteins. One role of CTLA-4 in inhibiting T cell responses seems to directly involve SHP-2 and PP2A dephosphorylation of T cell receptor-proximal signaling proteins, such as CD3 and LAT. CTLA-4 can also affect signaling indirectly, via competition with CD28 for CD80 and/or CD86 binding.

The first clinical evidence that modulation of T cell activation could result in effective anti-cancer therapy came from development of CTLA-4 blockade antibody ipilimumab.¹⁸ In some embodiments, ipilimumab is a human IgG1 antibody with specificity for CTLA-4. In some embodiments, another CTLA-4 blockade therapy, tremelimumab, is a human IgG2 antibody.

### PD-1

PD-1 is expressed on T cells, B cells, and certain myeloid cells; however, its role is best characterized in T cells. PD-1 expression on T cells is induced by antigen stimulation. Unlike CTLA-4, which limits early T-cell activation, PD-1 mainly exerts its inhibitory effect on T cells in the periphery where T cells encounter PD-1 ligands. Two ligands of PD-1 have been identified so far, PD-L1 and PD-L2, which are expressed by a large range of cell types, including tumor cells, monocyte-derived myeloid dendritic cells, epithelial cells, T cells, and B cells.¹⁸ In cancer, tumor cells and myeloid cells are thought to be main cell types mediating T-cell suppression through PD-1 ligation. It is still unclear whether effects of PD-L1 and PD-L2 on PD-1 downstream signaling are dependent on cell type that expresses a given ligand. Moreover, there are differences between PD-L1-versus PD-L2-induced effects, which remain to be fully elucidated.

Several mechanisms of PD-1-mediated T cell suppression have been proposed.¹⁸ One mechanism suggests that PD-1 ligation inhibits T cell activation only upon T cell receptor engagement. PD-1 has an intracellular “immunoreceptor tyrosine-based inhibition motif” or (ITIM) and an immunoreceptor tyrosine-based switch motif. It has been shown that PD-1 ligation leads to recruitment of phosphatases called “src homology 2 domain-containing tyrosine phosphatases,” or SHP-1 and SHP-2, to immunoreceptor tyrosine-based switch motif. Moreover, PD-1 ligation has been shown to interfere with signaling molecules, such as phosphatidylinositol-4,5-bisphosphate 3-kinase and Ras, which are important for T-cell proliferation, cytokine secretion, and metabolism. Analysis of human immunodeficiency virus (HIV)-specific T cells has also demonstrated PD-1-dependent basic leucine zipper transcription factor upregulation, which inhibits T cell function. Ligation of PD-1 has also been shown to induce metabolic alterations in T cells. Metabolic reprogramming of T cells from glycolysis to lipolysis is a consequence of PD-1-mediated impairment of T-cell effector function. Furthermore, PD-1-induced defects in mitochondrial respiration and glycolysis leads to impaired T-cell effector function that could be reversed by a mammalian target of rapamycin inhibition. Since most of the identified mechanisms of PD-1-mediated T-cell suppression are based on in vitro or ex vivo experiments, it remains to be demonstrated that these same mechanisms are responsible for T-cell exhaustion in vivo.

PD-1 is a type I membrane protein of 288 amino acids and is a member of the extended CD28/CTLA-4 family of T cell regulators.¹⁹ PD-1 protein structure includes an extracellular IgV domain followed by a transmembrane region and an intracellular tail, which contains two phosphorylation sites located in an immunoreceptor tyrosine-based inhibitory motif (ITIM) and an immunoreceptor tyrosine-based switch motif, which suggests that PD-1 negatively regulates T cell receptor signals. This is consistent with binding of SHP-1 and SHP-2 phosphatases to PD-1 cytoplasmic tail upon ligand binding. In addition, PD-1 ligation up-regulates E3-ubiquitin ligases CBL-b and c-CBL that trigger T cell receptor down-modulation. PD-1 is expressed on the surface of activated T cells, B cells, and macrophages, suggesting that compared to CTLA-4, PD-1 more broadly negatively regulates immune responses.

### CTLA-4 and PD-1 in Combination

Although monotherapies with CTLA-4- or PD-1-blocking antibodies have significantly prolonged survival of some patients with certain cancers, there are cases where some patients do not respond to therapy. A previous study has shown that combined treatment with ipilimumab (CTLA-4 blockade) and nivolumab (PD-1 blockade) induced better responses than either treatment alone.^{18,20} In some embodiments, immunotherapy, in accordance with the present disclosure, comprises both PD-1 blockade therapy and CTLA-4 blockade therapy. In certain embodiments, immunotherapy (e.g. immune checkpoint modulator therapy) involves treatment with an agent (e.g. antibody agent) that interferes with an interaction involving CTLA-4 and/or PD-1. In some embodiments, immunotherapy (e.g. immune checkpoint modulator therapy) involves administration of an agent (e.g. antibody agent) that specifically interacts with one or more of CTLA-4, CD80, CD86, PD-1 or PD-L1. In some embodiments, such therapy involves administration of one or more of atezolizumab, avelumab, durvalumab, ipilimumab, nivolumab, pembrolizumab, and/or tremelimumab.

### Tumor Mutational Load

Among other things, the present disclosure demonstrates that tumor mutational load can predict clinical efficacy of immunotherapy treatment for certain cancers. The present disclosure establishes, among other things, that in certain cases, individuals with higher tumor mutational loads are more likely to respond positively to immunotherapy than individuals with significantly lower tumor mutational loads. The present disclosure, among other things, establishes that, in certain cases, individuals with higher tumor mutational loads and who have already received immunotherapy, are more likely to respond positively to immunotherapy, than individuals with significantly lower tumor mutational loads.

In some embodiments, tumor mutational load comprises a number of somatic mutations within a region of a tumor genome. In some embodiments, somatic mutations comprise DNA alterations in non-germline cells and commonly occur in cancer cells. In some embodiments, a somatic mutation results in a neoantigen or neoepitope. It has been discovered that certain somatic mutations in cancer cells result in expression of neoepitopes, that in some embodiments transition a stretch of amino acids from being recognized as “self” to “non-self”. A cancer cell harboring a “non-self” antigen is typically more likely to elicit an immune response against a cancer cell. The identification of multiple mutations in a cancer sample as described herein can be useful for determining which cancer patients are likely to respond favorably to immunotherapy (e.g. continued and/or extended or modified immunotherapy); in some embodiments, such identification can be useful for determining which cancer patients are likely to respond, in particular, to treatment with an immune checkpoint modulator and/or otherwise to PD-1 and/or CTLA-4 blockade.

The present disclosure, among other things, demonstrates that, for certain cancers, patients with high numbers of somatic mutations, or a high tumor mutational load, are more likely to benefit from treatment with immune checkpoint modulators than those patients with lower tumor mutational loads. In some embodiments, patients with a high tumor mutational load respond better to PD-1 (programmed cell death 1) blockade than those patients with a significantly lower tumor mutational load. In some embodiments, individuals with a high tumor mutational load respond better to treatment with anti-PD-1 antibodies than those individuals with a low tumor mutational load. In some embodiments, individuals with a high tumor mutational load respond better to treatment with CTLA-4 blockade than those individuals with a low tumor mutational load. In some embodiments, individuals with a high tumor mutational load respond better to treatment with CTLA-4 antibodies than those individuals with a low tumor mutational load.

### Cancers

In some embodiments, the present disclosure relates to treatment of cancer. Certain exemplary cancers that may, in some embodiments, be treated in accordance with the present disclosure include, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

In some embodiments, a cancer may involve one or more tumors. In some embodiments, a tumor comprises a solid tumor. In some embodiments, the present disclosure relates to treatment of melanoma and non-small cell lung cancer.

### Administration

In accordance with certain methods of the invention, an immune checkpoint modulator is or has been administered to an individual. In some embodiments, treatment with an immune checkpoint modulator is utilized as a sole therapy. In some embodiments, treatment with an immune checkpoint modulator is used in combination with one or more other therapies.

Those of ordinary skill in the art will appreciate that appropriate formulations, indications, and dosing regimens are typically analyzed and approved by government regulatory authorities such as the Food and Drug Administration in the United States. In many embodiments, an immune checkpoint modulator is administered in accordance with the present invention according to an approved protocol. However, the present disclosure provides certain technologies for identifying, characterizing, and/or selecting particular patients to whom immune checkpoint modulators may desirably be administered. In some embodiments, insights provided by the present disclosure permit dosing of a given immune checkpoint modulator with greater frequency and/or greater individual doses (e.g., due to reduced susceptibility to and/or incidence or intensity of undesirable effects) relative to that recommended or approved based on population studies that include both individuals identified as described herein and other individuals. In some embodiments, insights provided by the present disclosure permit dosing of a given immune checkpoint modulator with reduced frequency and/or reduced individual doses (e.g., due to increased responsiveness) relative to that recommended or approved based on population studies that include both individuals identified as described herein and other individuals.

In some embodiments, an immune system modulator is administered in a pharmaceutical composition that also comprises a physiologically acceptable carrier or excipient. In some embodiments, a pharmaceutical composition is sterile. In many embodiments, a pharmaceutical composition is formulated for a particular mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, etc., as well as combinations thereof. A pharmaceutical preparation can, if desired, comprise one or more auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used.

In some embodiments, a pharmaceutical composition or medicament, if desired, can contain an amount (typically a minor amount) of wetting or emulsifying agents, and/or of pH buffering agents. In some embodiments, a pharmaceutical composition can be a liquid solution, suspension, emulsion, tablet, pill, capsule, sustained release formulation, or powder. In some embodiments, a pharmaceutical composition can be formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

In some embodiments, a pharmaceutical composition can be formulated in accordance with the routine procedures as a pharmaceutical composition adapted for administration to human beings. For example, in some embodiments, a composition for intravenous administration typically is a solution in sterile isotonic aqueous buffer. Where necessary, a composition may also include a solubilizing agent and a local anesthetic to ease pain at the site of the injection. Generally, ingredients are supplied either separately or mixed together in unit dosage form, for example, as a dry lyophilized powder or water free concentrate in a hermetically sealed container such as an ampule or sachet indicating the quantity of active agent. Where a composition is to be administered by infusion, it can be dispensed with an infusion bottle containing sterile pharmaceutical grade water, saline or dextrose/water. Where a composition is administered by injection, an ampule of sterile water for injection or saline can be provided so that the ingredients may be mixed prior to administration.

In some embodiments, an immune checkpoint modulator can be formulated in a neutral form; in some embodiments it may be formulated in a salt form. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Pharmaceutical compositions for use in accordance with the present invention may be administered by any appropriate route. In some embodiments, a pharmaceutical composition is administered intravenously. In some embodiments, a pharmaceutical composition is administered subcutaneously. In some embodiments, a pharmaceutical composition is administered by direct administration to a target tissue, such as heart or muscle (e.g., intramuscular), or nervous system (e.g., direct injection into the brain; intraventricularly; intrathecally). Alternatively or additionally, in some embodiments, a pharmaceutical composition is administered parenterally, transdermally, or transmucosally (e.g., orally or nasally). More than one route can be used concurrently, if desired.

Immune checkpoint modulators (or a composition or medicament containing an immune checkpoint modulator, can be administered alone, or in conjunction with other immune checkpoint modulators. The term, “in conjunction with,” indicates that a first immune checkpoint modulator is administered prior to, at about the same time as, or following another immune checkpoint modulator. For example, a first immune checkpoint modulator can be mixed into a composition containing one or more different immune checkpoint modulators, and thereby administered contemporaneously; alternatively, the agent can be administered contemporaneously, without mixing (e.g., by “piggybacking” delivery of the agent on the intravenous line by which the immune checkpoint modulator is also administered, or vice versa). In another example, the immune checkpoint modulator can be administered separately (e.g., not admixed), but within a short time frame (e.g., within 24 hours) of administration of the immune checkpoint modulator.

In some embodiments, subjects treated with immune checkpoint modulators are administered one or more immunosuppressants. In some embodiments, one or more immunosuppressants are administered to decrease, inhibit, or prevent an undesired autoimmune response (e.g., enterocolitis, hepatitis, dermatitis (including toxic epidermal necrolysis), neuropathy, and/or endocrinopathy), for example, hypothyroidism. Exemplary immunosuppressants include steroids, antibodies, immunoglobulin fusion proteins, and the like. In some embodiments, an immunosuppressant inhibits B cell activity (e.g. rituximab). In some embodiments, an immunosuppressant is a decoy polypeptide antigen.

In some embodiments, immune checkpoint modulators (or a composition or medicament containing immune checkpoint modulators) are administered in a therapeutically effective amount (e.g., a dosage amount and/or according to a dosage regimen that has been shown, when administered to a relevant population, to be sufficient to treat cancer, such as by ameliorating symptoms associated with the cancer, preventing or delaying the onset of the cancer, and/or also lessening the severity or frequency of symptoms of cancer). In some embodiments, long term clinical benefit is observed after treatment with immune checkpoint modulators, including, for example, PD-1 blockers such as pembrolizumab, and/or other agents. Those of ordinary skill in the art will appreciate that a dose which will be therapeutically effective for the treatment of cancer in a given patient may depend, at least to some extent, on the nature and extent of cancer, and can be determined by standard clinical techniques. In some embodiments, one or more in vitro or in vivo assays may optionally be employed to help identify optimal dosage ranges. In some embodiments, a particular dose to be employed in the treatment of a given individual may depend on the route of administration, the extent of cancer, and/or one or more other factors deemed relevant in the judgment of a practitioner in light of patient's circumstances. In some embodiments, effective doses may be extrapolated from dose-response curves derived from in vitro or animal model test systems (e.g., as described by the U.S. Department of Health and Human Services, Food and Drug Administration, and Center for Drug Evaluation and Research in “Guidance for Industry: Estimating Maximum Safe Starting Dose in Initial Clinical Trials for Therapeutics in Adult Healthy Volunteers”, Pharmacology and Toxicology, July 2005.

In some embodiments, a therapeutically effective amount of an immune check point modulator can be, for example, more than about 0.01 mg/kg, more than about 0.05 mg/kg, more than about 0.1 mg/kg, more than about 0.5 mg/kg, more than about 1.0 mg/kg, more than about 1.5 mg/kg, more than about 2.0 mg/kg, more than about 2.5 mg/kg, more than about 5.0 mg/kg, more than about 7.5 mg/kg, more than about 10 mg/kg, more than about 12.5 mg/kg, more than about 15 mg/kg, more than about 17.5 mg/kg, more than about 20 mg/kg, more than about 22.5 mg/kg, or more than about 25 mg/kg body weight. In some embodiments, a therapeutically effective amount can be about 0.01-25 mg/kg, about 0.01-20 mg/kg, about 0.01-15 mg/kg, about 0.01-10 mg/kg, about 0.01-7.5 mg/kg, about 0.01-5 mg/kg, about 0.01-4 mg/kg, about 0.01-3 mg/kg, about 0.01-2 mg/kg, about 0.01-1.5 mg/kg, about 0.01-1.0 mg/kg, about 0.01-0.5 mg/kg, about 0.01-0.1 mg/kg, about 1-20 mg/kg, about 4-20 mg/kg, about 5-15 mg/kg, about 5-10 mg/kg body weight. In some embodiments, a therapeutically effective amount is about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, body weight, or more. In some embodiments, the therapeutically effective amount is no greater than about 30 mg/kg, no greater than about 20 mg/kg, no greater than about 15 mg/kg, no greater than about 10 mg/kg, no greater than about 7.5 mg/kg, no greater than about 5 mg/kg, no greater than about 4 mg/kg, no greater than about 3 mg/kg, no greater than about 2 mg/kg, or no greater than about 1 mg/kg body weight or less.

In some embodiments, the administered dose for a particular individual is varied (e.g., increased or decreased) over time, depending on the needs of the individual.

In yet another example, a loading dose (e.g., an initial higher dose) of a therapeutic composition may be given at the beginning of a course of treatment, followed by administration of a decreased maintenance dose (e.g., a subsequent lower dose) of the therapeutic composition.

Without wishing to be bound by any theories, it is contemplated that a loading dose may clear out an initial and, in some cases massive, accumulation of undesirable materials (e.g., fatty materials and/or tumor cells, etc.) in tissues (e.g., in the liver), and maintenance dosing may delay, reduce, or prevent buildup of fatty materials after initial clearance.

It will be appreciated that a loading dose and maintenance dose amounts, intervals, and duration of treatment may be determined by any available method, such as those exemplified herein and those known in the art. In some embodiments, a loading dose amount is about 0.01-1 mg/kg, about 0.01-5 mg/kg, about 0.01-10 mg/kg, about 0.1-10 mg/kg, about 0.1-20 mg/kg, about 0.1-25 mg/kg, about 0.1-30 mg/kg, about 0.1-5 mg/kg, about 0.1-2 mg/kg, about 0.1-1 mg/kg, or about 0.1-0.5 mg/kg body weight. In some embodiments, a maintenance dose amount is about 0-10 mg/kg, about 0-5 mg/kg, about 0-2 mg/kg, about 0-1 mg/kg, about 0-0.5 mg/kg, about 0-0.4 mg/kg, about 0-0.3 mg/kg, about 0-0.2 mg/kg, about 0-0.1 mg/kg body weight. In some embodiments, a loading dose is administered to an individual at regular intervals for a given period of time (e.g., 1, 2, 3, 4, 5, 6, 7, 8, 9, 10, 11, 12 or more months) and/or a given number of doses (e.g., 1, 2, 3, 4, 5, 6, 7, 8, 9, 10, 15, 20, 25, 30 or more doses), followed by maintenance dosing. In some embodiments, a maintenance dose ranges from 0-2 mg/kg, about 0-1.5 mg/kg, about 0-1.0 mg/kg, about 0-0.75 mg/kg, about 0-0.5 mg/kg, about 0-0.4 mg/kg, about 0-0.3 mg/kg, about 0-0.2 mg/kg, or about 0-0.1 mg/kg body weight. In some embodiments, a maintenance dose is about 0.01, 0.02, 0.04, 0.06, 0.08, 0.1, 0.2, 0.3, 0.4, 0.5, 0.6, 0.7, 0.8, 0.9, 1.0, 1.2, 1.4, 1.6, 1.8, or 2.0 mg/kg body weight. In some embodiments, maintenance dosing is administered for 1, 2, 3, 4, 5, 6, 7, 8, 9, 10, 11, 12 or more months. In some embodiments, maintenance dosing is administered for 1, 2, 3, 4, 5, 6, 7, 8, 9, 10 or more years. In some embodiments, maintenance dosing is administered indefinitely (e.g., for life time).

A therapeutically effective amount of an immune checkpoint modulator may be administered as a one-time dose or administered at intervals, depending on the nature and extent of the cancer, and on an ongoing basis. Administration at an “interval,” as used herein indicates that the therapeutically effective amount is administered periodically (as distinguished from a one-time dose). The interval can be determined by standard clinical techniques. In some embodiments, an immune checkpoint modulator is administered bimonthly, monthly, twice monthly, triweekly, biweekly, weekly, twice weekly, thrice weekly, or daily. The administration interval for a single individual need not be a fixed interval, but can be varied over time, depending on the needs and rate of recovery of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The invention additionally pertains to a pharmaceutical composition comprising an immune checkpoint modulator, as described herein, in a container (e.g., a vial, bottle, bag for intravenous administration, syringe, etc.) with a label containing instructions for administration of the composition for treatment of cancer.

### Combination Therapy

In some embodiments, an immunomodulatory agent can be used in combination with another therapeutic agent to treat diseases such as cancer. In some embodiments, an immunomodulatory agent, or a pharmaceutical composition comprising immunotherapy as described herein can optionally contain, and/or be administered in combination with, one or more additional therapeutic agents, such as a cancer therapeutic agent, e.g., a chemotherapeutic agent or a biological agent. An additional agent can be, for example, a therapeutic agent that is art-recognized as being useful to treat the disease or condition being treated by the immunomodulatory agent, e.g., an anti-cancer agent, or an agent that ameliorates a symptom associated with the disease or condition being treated. The additional agent also can be an agent that imparts a beneficial attribute to the therapeutic composition (e.g., an agent that affects the viscosity of the composition). In some embodiments, the additional agent can be a kinase inhibitor. In some embodiments, immunotherapy is administered to a subject who has received, is receiving, and/or will receive therapy with another therapeutic agent or modality (e.g., with a chemotherapeutic agent, surgery, radiation, or a combination thereof).

Some embodiments of combination therapy modalities provided by the present disclosure provide, for example, administration of an immunomodulatory agent and additional agent(s) in a single pharmaceutical formulation. Some embodiments provide administration of an immunomodulatory agent and administration of an additional therapeutic agent in separate pharmaceutical formulations.

Examples of chemotherapeutic agents that can be used in combination with an immunomodulatory agent described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods described herein include monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, BMS-936559, RG7446/MPDL3280A, MEDI4736, tremelimumab, or others known in the art), enzymes (e.g., L-asparaginase), cytokines (e.g., interferons and interleukins), growth factors (e.g., colony stimulating factors and erythropoietin), cancer vaccines, gene therapy vectors, or any combination thereof.

In some embodiments, an immunomodulatory agent is administered to a subject in need thereof in combination with another agent for the treatment of cancer, either in the same or in different pharmaceutical compositions. In some embodiments, the additional agent is an anticancer agent. In some embodiments, the additional agent affects (e.g., inhibits) histone modifications, such as histone acetylation or histone methylation. In certain embodiments, an additional anticancer agent is selected from the group consisting of chemotherapeutics (such as 2CdA, 5-FU, 6-Mercaptopurine, 6-TG, Abraxane™ Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda®, and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Velcade®, and Zevalin™); small molecules (such as Tykerb®); corticosteroids (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as Iodotope®, Metastron®, Phosphocol® and Samarium SM-153).

The additional agents that can be used in combination with immunotherapy as set forth above are for illustrative purposes and not intended to be limiting. The combinations embraced by this disclosure, include, without limitation, one or more immunomodulatory agent(s) as provided herein or otherwise known in the art, and at least one additional agent selected from the lists above or otherwise provided herein. Immunomodulatory agents can also be used in combination with one or with more than one additional agent, e.g., with two, three, four, five, or six, or more, additional agents.

In some embodiments, treatment methods described herein are performed on subjects for which other treatments of the medical condition have failed or have had less success in treatment through other means, e.g., in subjects having a cancer refractory to standard-of-care treatment. Additionally, the treatment methods described herein can be performed in conjunction with one or more additional treatments of the medical condition, e.g., in addition to or in combination with standard-of-care treatment. For instance, the method can comprise administering a cancer regimen, e.g., nonmyeloablative chemotherapy, surgery, hormone therapy, and/or radiation, prior to, substantially simultaneously with, or after the administration of an immunomodulatory agent described herein, or composition thereof. In certain embodiments, a subject to which an immunomodulatory agent described herein is administered can also be treated with antibiotics and/or one or more additional pharmaceutical agents.

### EXEMPLIFICATION

### Example 1: Evolutionary Divergence of HLA-I Genotype Impacts Efficacy of Cancer Therapy

Functional diversity of major histocompatibility complex class I (MHC-I) molecules, encoded by the highly polymorphic human leukocyte antigen class I (HLA-I) genes, underlies successful immunologic control of both infectious disease and cancer. The divergent allele advantage hypothesis dictates that a HLA-I genotype with two alleles whose sequences are more divergent enables presentation of a more diverse immunopeptidome. However, the effect of sequence divergence between HLA-I alleles—a quantifiable measure of HLA-I evolution—on the efficacy of immune checkpoint inhibitor (ICI) treatment for cancer remains unknown. Here, we determined the germline HLA-I evolutionary divergence (HED) of patients with melanoma and non-small cell lung cancer treated with ICI, by quantifying the physiochemical sequence divergence between HLA-I alleles of each patient's genotype.

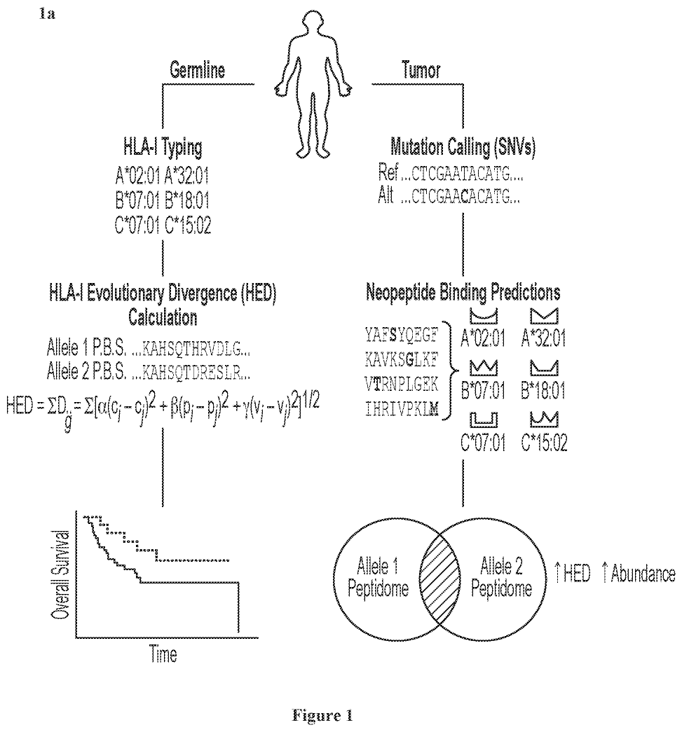

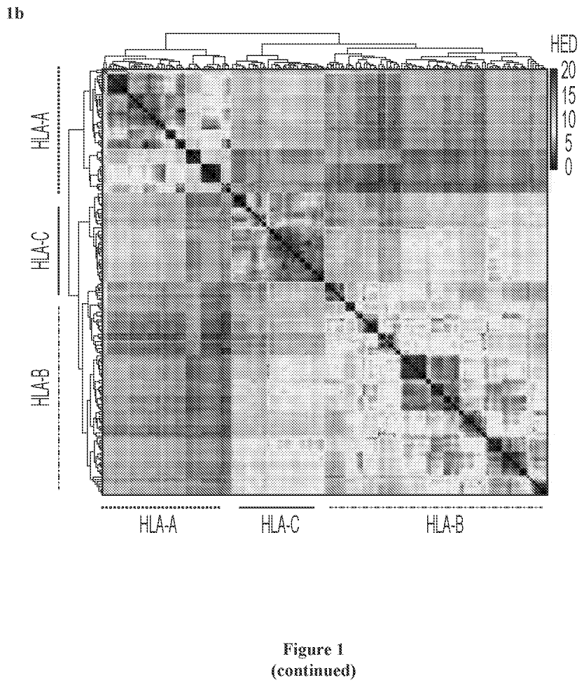

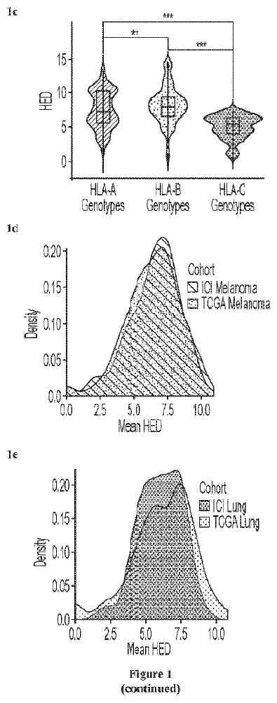

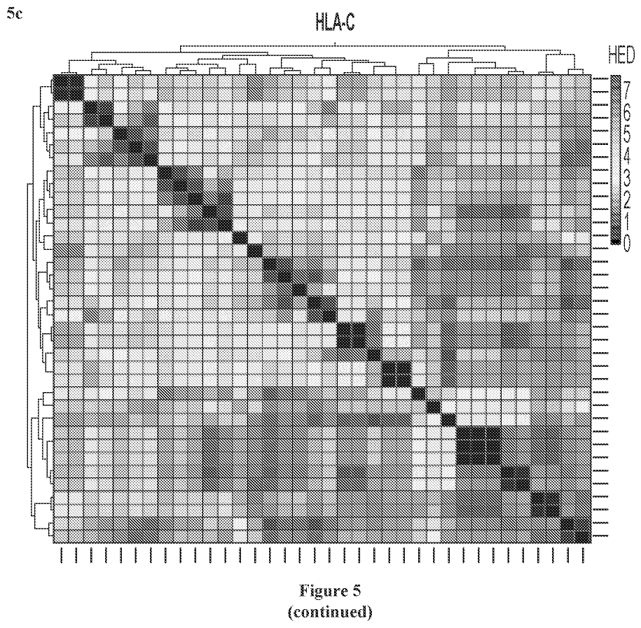

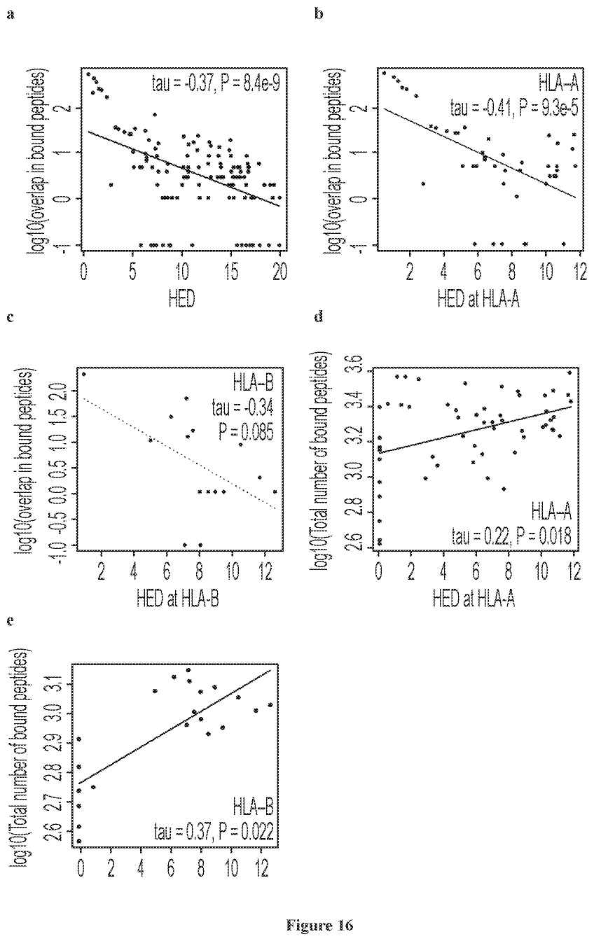

We first determined HLA-I evolutionary divergence (HED) using HLA-I genotypes across multiple patient cohorts with metastatic melanoma or non-small cell lung cancer (NSCLC) treated with CTLA-4 blockade or PD-1/PD-L1 blockade (FIG. 1a, Table 2). For each patient, we calculated HED at each of HLA-A, HLA-B, and HLA-C by measuring the Grantham distance^{32,34} between the peptide-binding domains of the two alleles. The Grantham distance is a classic metric that allows quantification of physiochemical differences between protein amino acid sequences, taking into account composition, polarity, and volume. To explore the landscape of HEDs in our dataset, we performed hierarchical clustering of HED per locus for all pairwise allele combinations across HLA-A, HLA-B, and HLA-C. Hierarchical clustering of HEDs from each locus demonstrated distinct clusters of high and low divergence between alleles (FIG. 1b, FIG. 5 a-5c), as expected and consistent with known relationships between HLA-A, HLA-B, and HLA-C loci^{26,35}. It also showed that HLA-B pairwise divergences are higher relative to HLA-A and HLA-C (FIG. 1c), consistent with prior reports that HLA-B is the oldest and most diverse of the three HLA-I loci^{23,35}. Moreover, HLA-C alleles had the lowest pairwise divergences, in line with prior studies that HLA-C has evolved most recently^{23,35} (Buhler, S., Nunes, J. M. & Sanchez-Mazas, A. HLA class I molecular variation and peptide-binding properties suggest a model of joint divergent asymmetric selection. Immunogenetics 68, 401-416 (2016); FIG. 1c). For each patient, we next calculated the mean HED as the mean of the three pairwise divergences of HLA-A, HLA-B, and HLA-C, assuming that each locus contributes equally to presentation of antigenic peptides. Mean HED distributions in patients from our cohorts were similar to those observed in the TCGA cohorts (FIG. 1d,e). A prior comparison of the Grantham distance to other common metrics of sequence divergence showed that the Grantham distance best captured the functional properties of HLA-I molecules³². The Grantham distance is a well-recognized metric that has been applied to measure amino acid polymorphism in many studies of comparative evolution, cancer, infectious disease, and immunity³⁶⁻⁴². Furthermore, in an analysis of HLA-I allele pairs and naturally eluted peptides derived from mass spectrometry and monoallelic cell lines, we detected an association between HED and peptidome diversity (Abelin, J. G., et al. Mass Spectrometry Profiling of HLA-Associated Peptidomes in Mono-allelic Cells Enables More Accurate Epitope Prediction. Immunity 46, 315-326 (2017); FIG. 16). Taken together, these data verify that the Grantham distance is a suitable measure of HLA-I polymorphism in our patient cohorts.

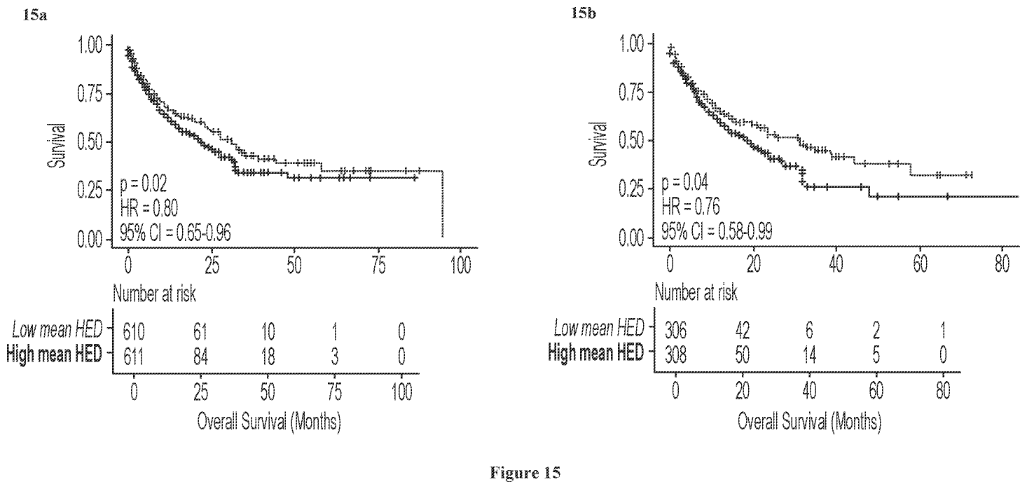

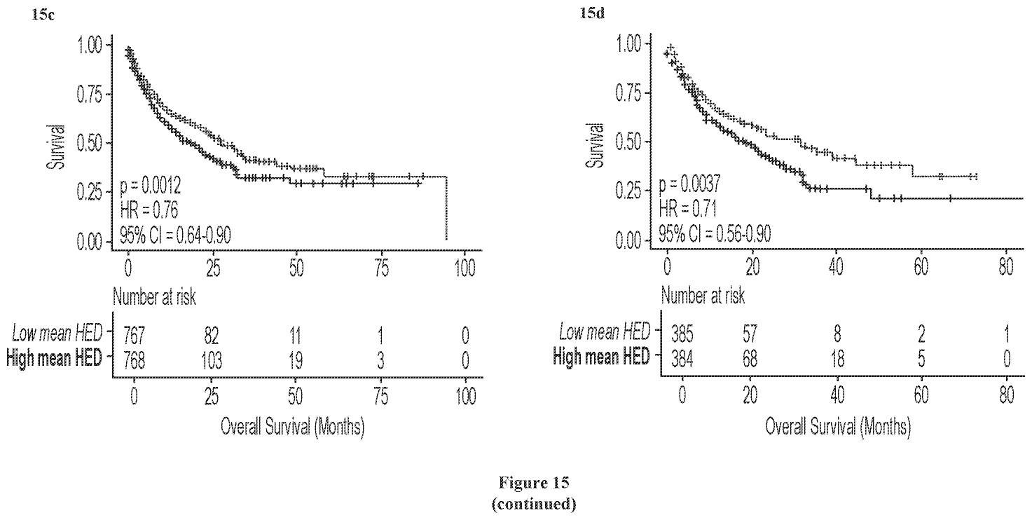

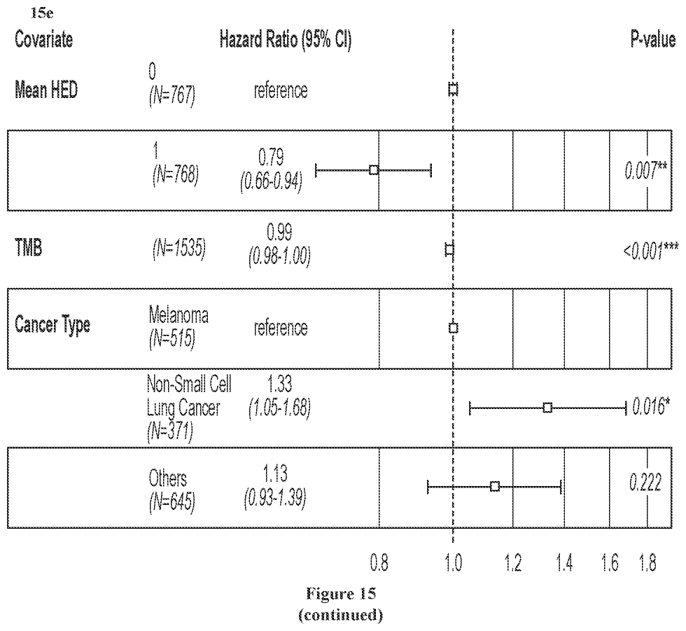

TABLE 2
Table shows cohorts and references for all patients
included in the analysis. All patients analyzed in FIG. 15
are described in Chowell et al Science 2018.

| | Number | Type of | Cancer | |
|---|---|---|---|---|
| Study | of Patients | Sequencing | Type | Drug Class |
| Van Alien et al. | 100 | Whole-exome | Melanoma | CTLA-4 |
| Science 2015 | | | | |
| Rizvi et al. | 100 | Whole exome for | NSCLC | PD-1, |
| Science 2015 + | | 34/100; 66/100 | | PD-L1, |
| Chowell et al. | | received HLA-I | | CTLA-4, |
| Science 2018 | | molecular typing | | combination |
| Chowell et al. | 114 | Targeted panel | Melanoma | PD-1/ |
| Science 2018 | | | | PD-L1 |

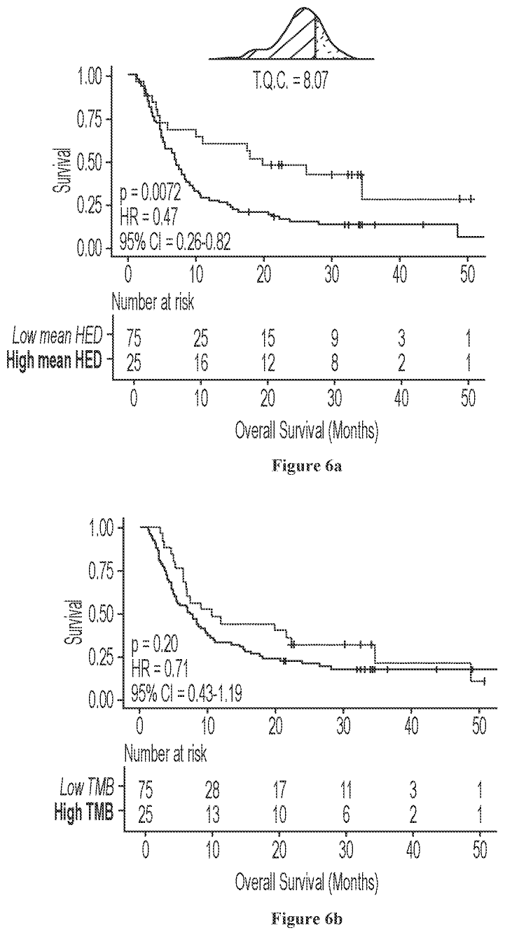

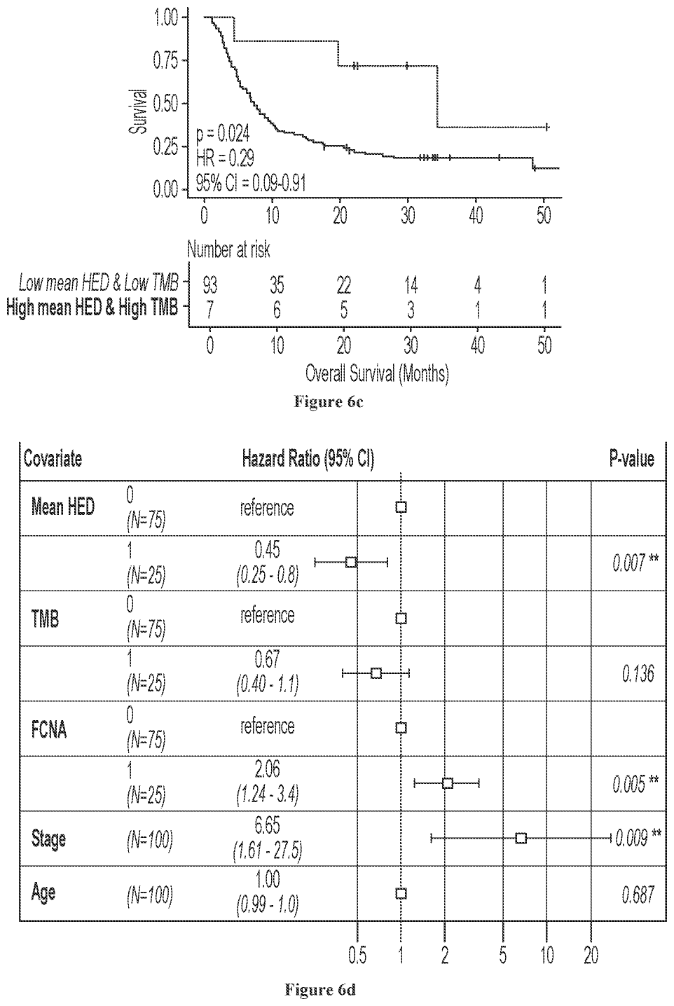

We next asked whether HED is associated with response to ICI. We stratified patients by mean HED in a cohort of 100 patients with melanoma treated with anti-CTLA-48 (hereafter called cohort 1), and observed improved overall survival after ICI therapy in patients with high mean HED (P=0.0072, HR=0.47, 95% confidence interval (C.I.)=0.26-0.82) (FIG. 6a). These results were similar across different metrics (i.e. sum, median, or geometric mean) used to combine pairwise divergences of HLA-A, HLA-B, and HLA-C alleles (Table 1). We also found that the effect of mean HED on survival was independent of tumor mutational burden (TMB) and other relevant genomic and clinical variables, when these were included in a multivariable Cox regression model of survival (FIG. 6d). Finally, we found that the effect of both high mean HED and high TMB on overall survival after ICI was more pronounced than the effect of either alone, as reflected by the reduction in hazard ratio (commonly considered to be the effect size in survival analyses; Bradburn, M. J., Clark, T. G., Love, S. B. & Altman, D. G. Survival analysis part II: multivariate data analysis—an introduction to concepts and methods. Br J Cancer 89, 431-436 (2003); Brostrom, G.r. Event history analysis with R, (CRC Press, Boca Raton, Fla., 2012)) when considering both variables (FIG. 6a-6c).

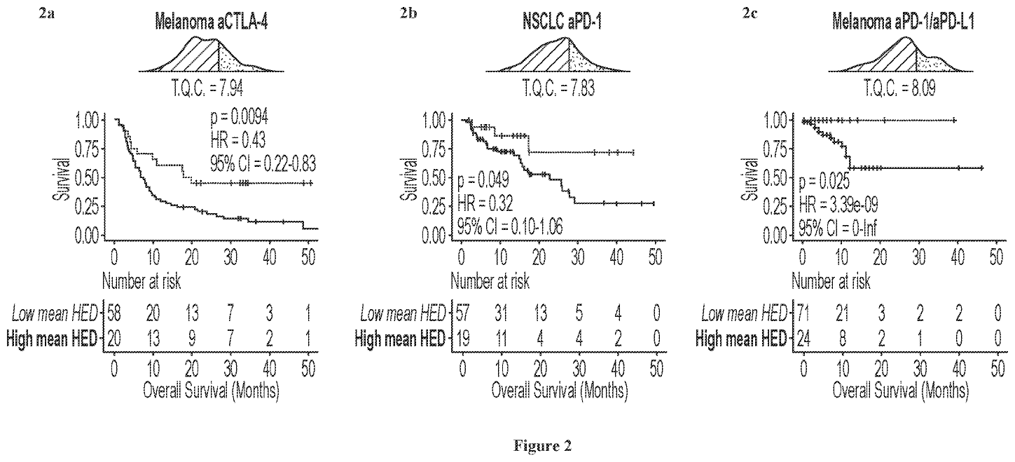

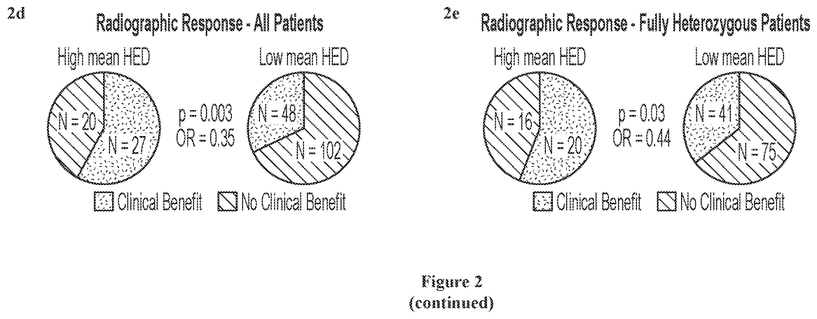

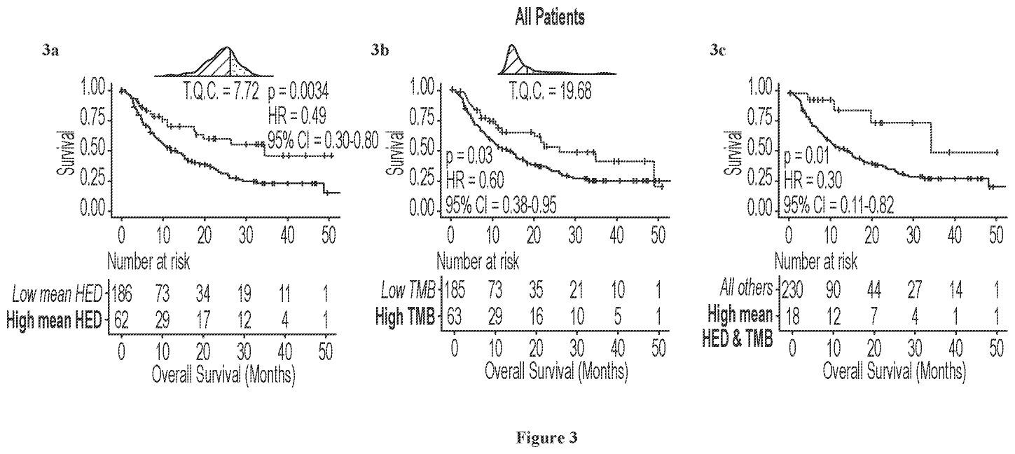

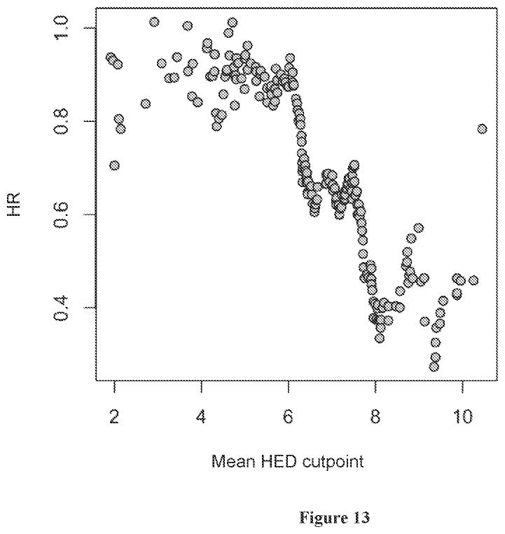

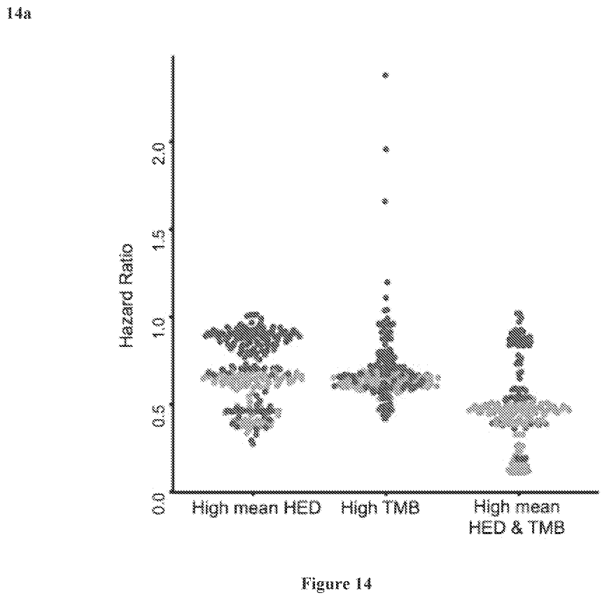

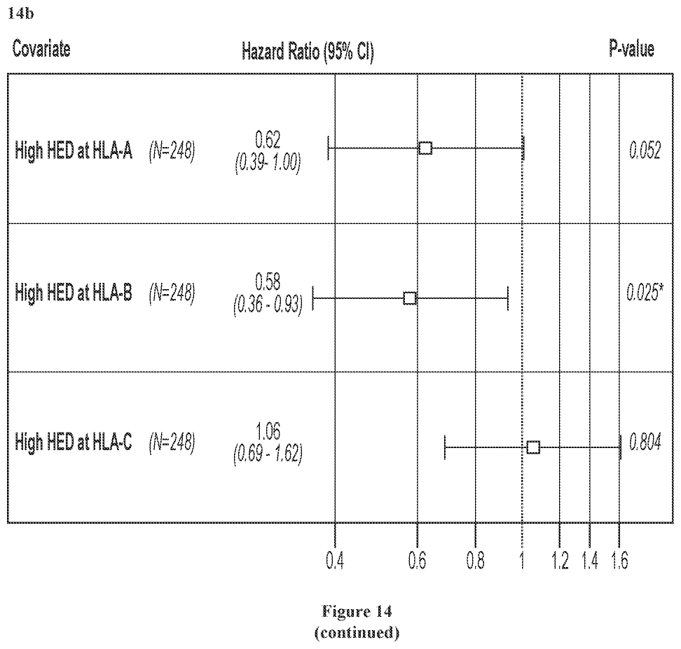

Prior studies of divergent allele advantage have suggested that the diversity of peptide repertoires of fully heterozygous HLA-I genotypes varies with sequence divergence^{20,30,32}. Therefore, we hypothesized that even among patients fully heterozygous at HLA-I, response to ICI may also vary with HED. Strikingly, we found that high mean HED was associated with improved survival after ICI in the 78 fully heterozygous patients from cohort 18 (P=0.0094, HR=0.43, 95% C.I.=0.22-0.83) (FIG. 2a). In a second cohort of 76 fully heterozygous patients with NSCLC treated with anti-PD-1^{5,14}, we also found that high mean HED was associated with better overall survival (P=0.049, HR=0.32, 95% C.I.=0.10-1.06) (FIG. 2b). We observed the same in an additional third cohort of 95 fully heterozygous patients with metastatic melanoma treated with anti-PD-1^{14,43}(P=0.025) (FIG. 2c). In a combined analysis of all three cohorts, we noted a negative relationship between mean HED and hazard ratio, indicating that in general, an increase in mean HED corresponds to improved overall survival (FIG. 13). Beyond survival, clinical response to ICI was also associated with high mean HED when considering all patients (HLA-I homozygotes or heterozygotes) (57.4% vs. 32.0%, P=0.003, OR=0.35) (FIG. 2d), or only fully heterozygous patients (55.6% vs. 35.3%, P=0.03, OR=0.44) (FIG. 2e) in all cohorts.

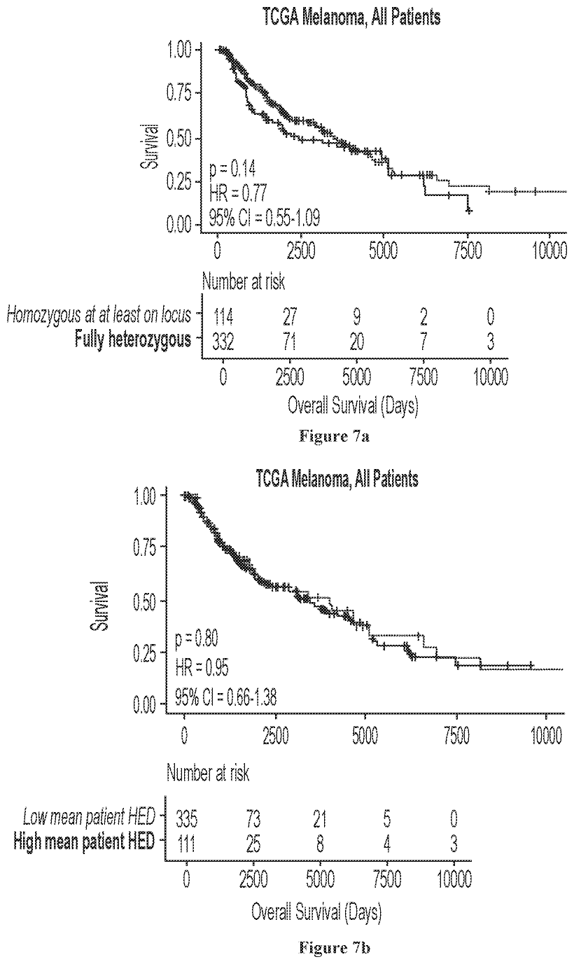

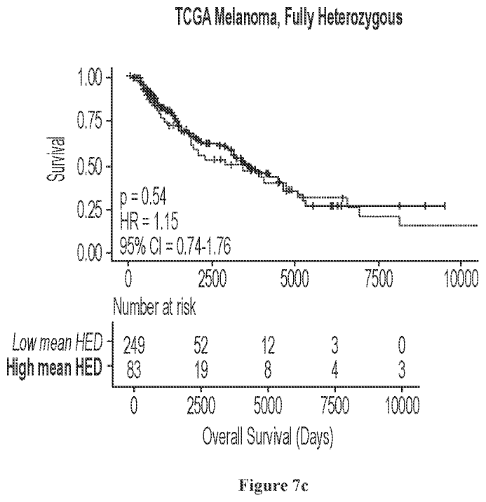

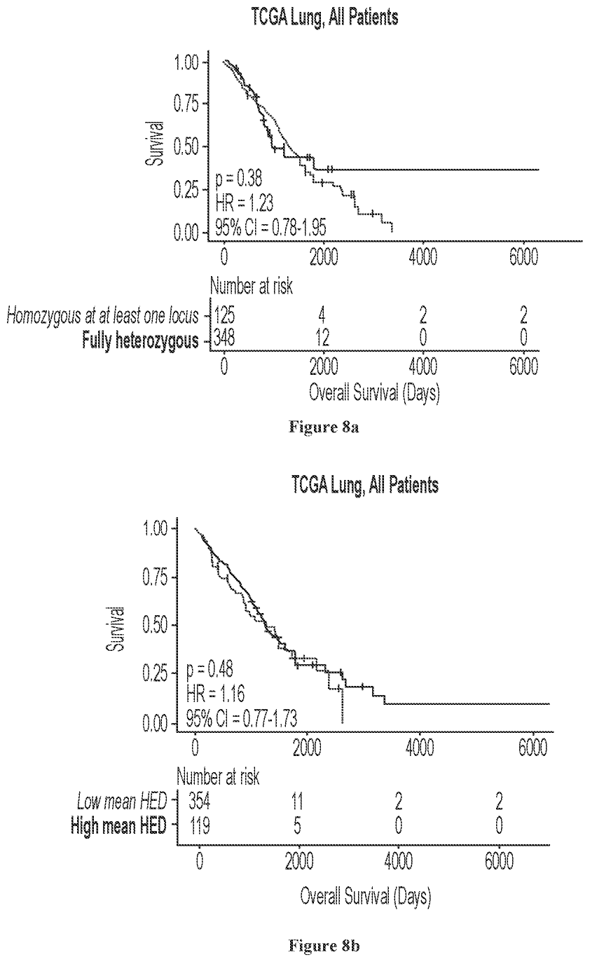

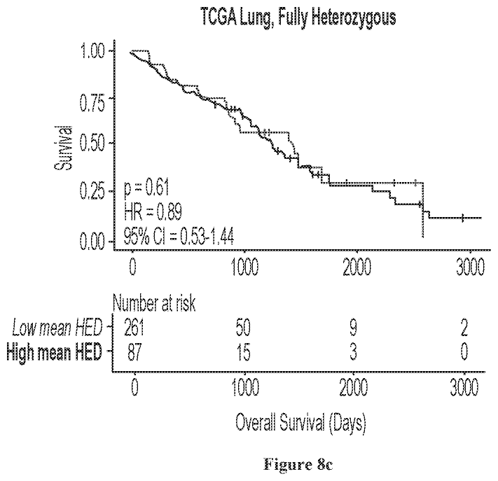

These data indicate that HED is predictive of survival and response among cancer patients treated with ICI. To determine whether HED might simply reflect a general prognostic factor in cancer, we examined the association of HLA-I heterozygosity or HED with overall survival among patients with melanoma and non-small cell lung cancer who did not receive ICI therapy, and whose tumors were profiled with exome sequencing, and observed no effect (FIG. 7, 8). This suggests that mean HED is predictive of response to ICI, and not prognostic.

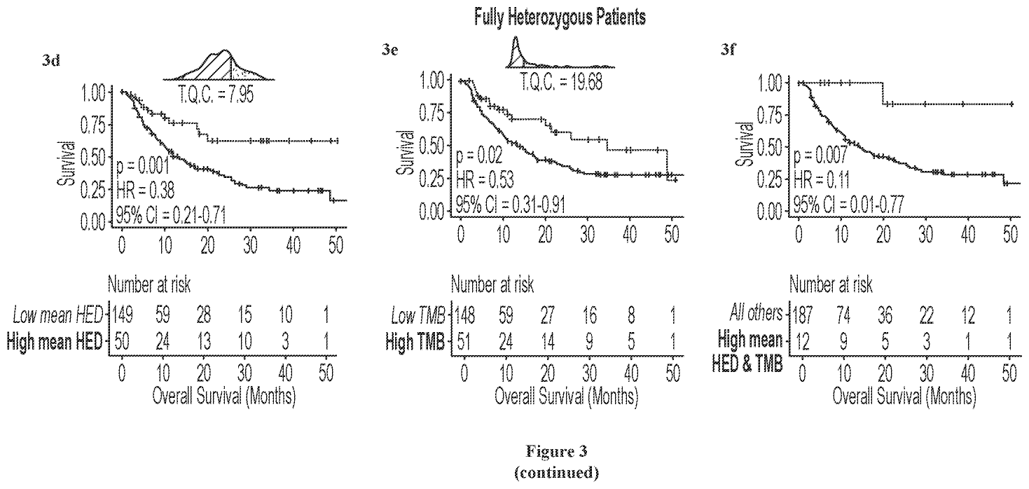

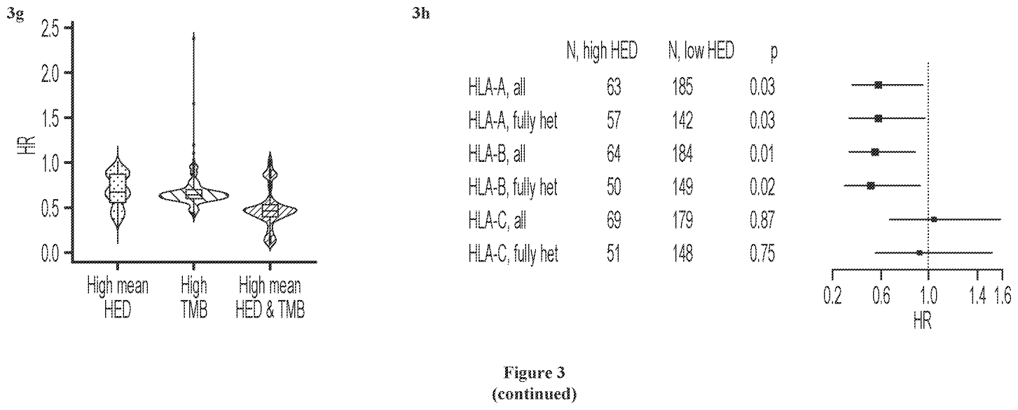

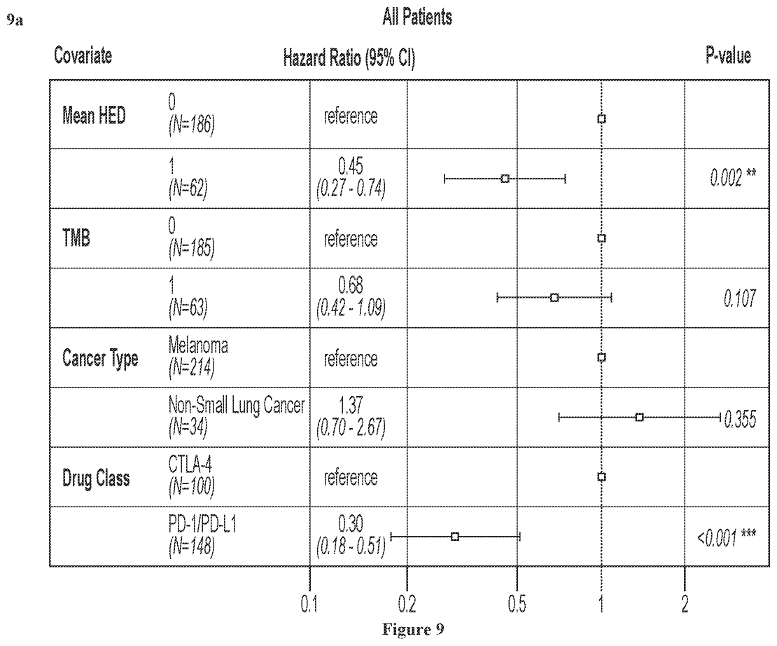

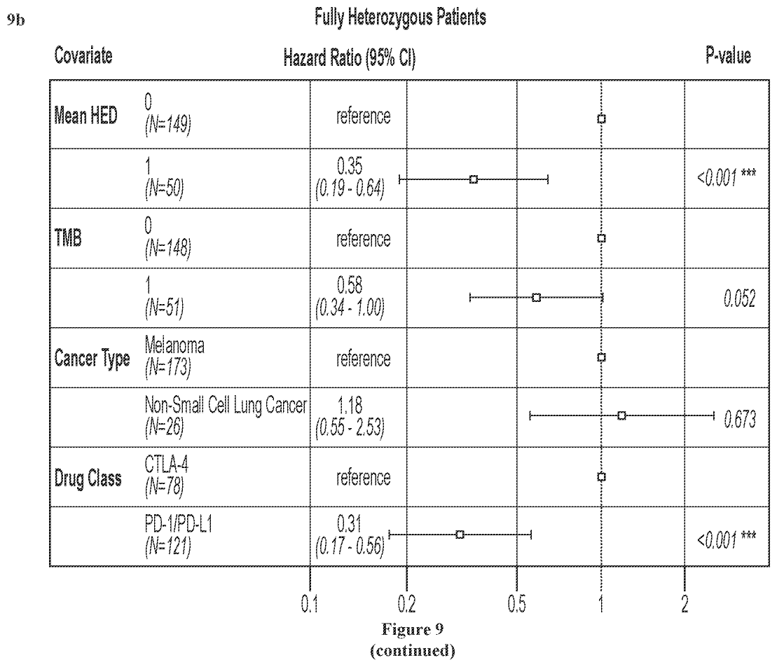

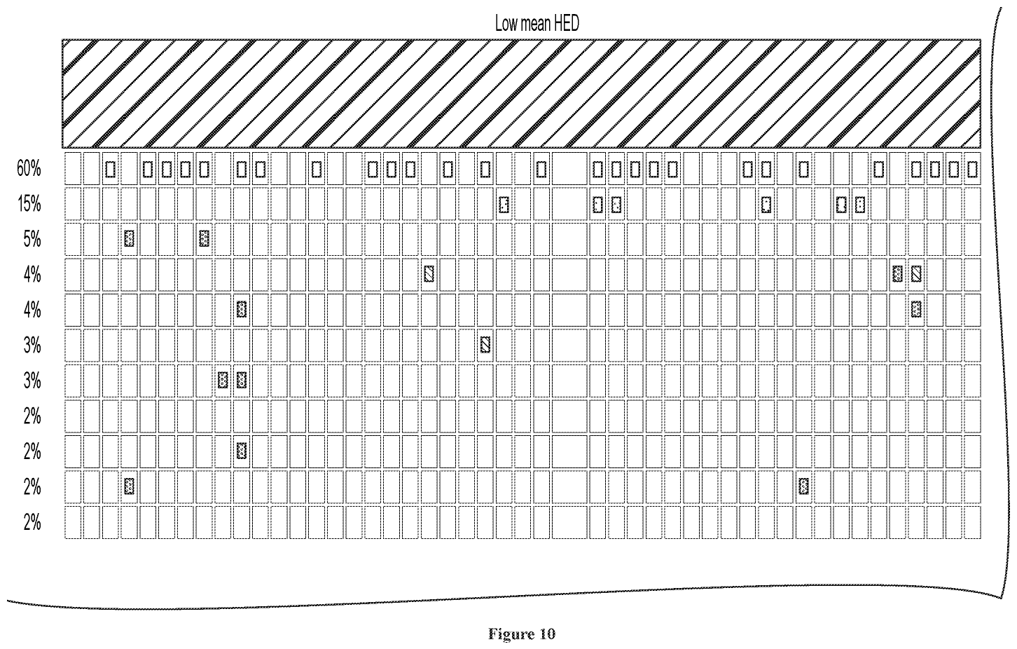

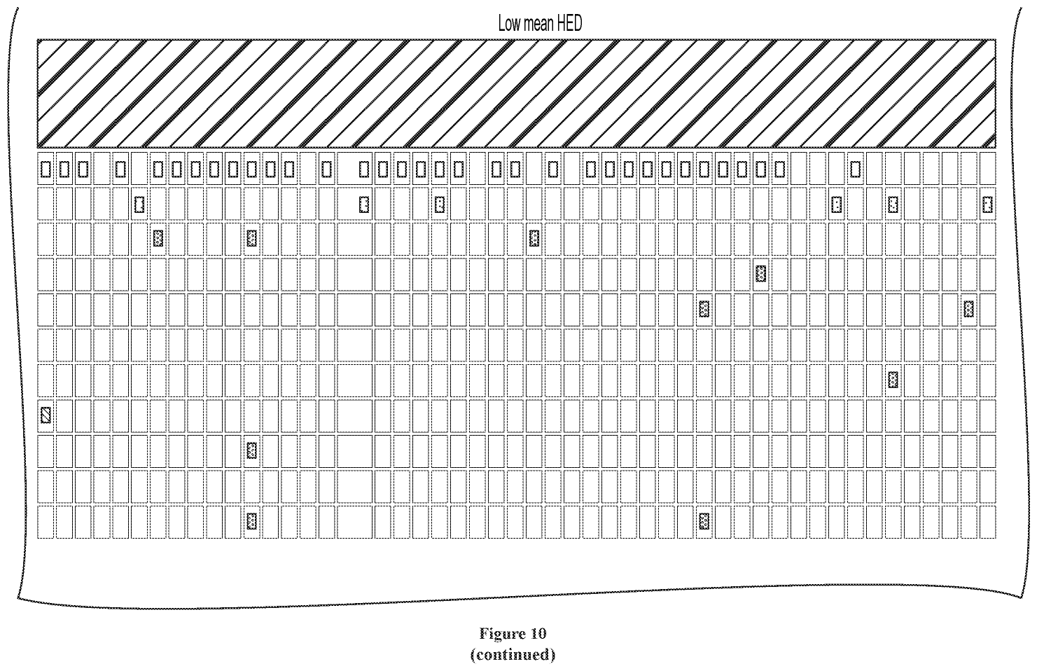

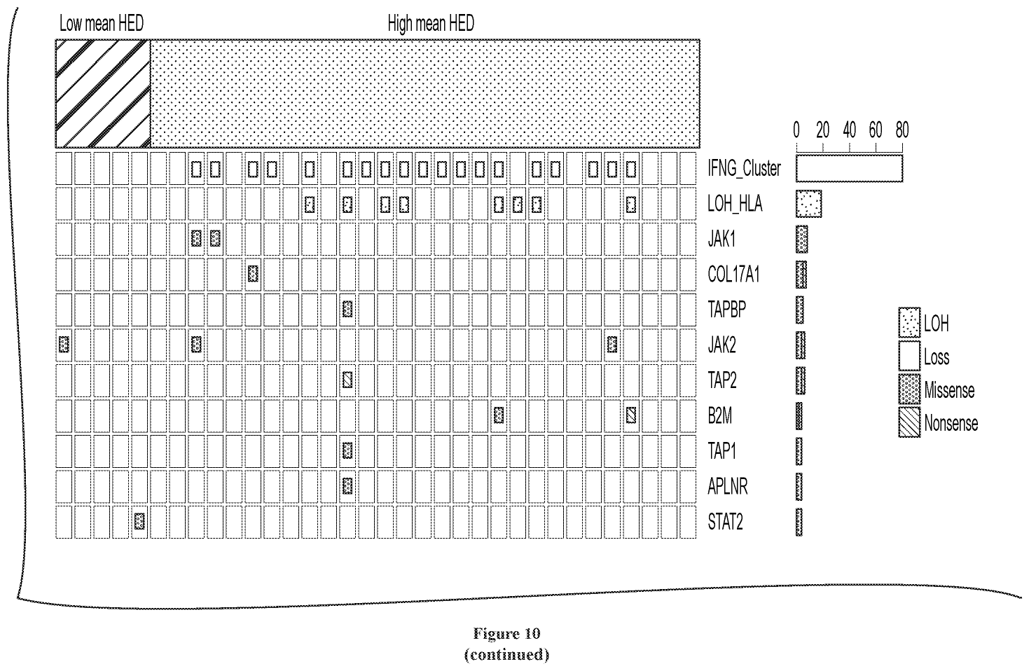

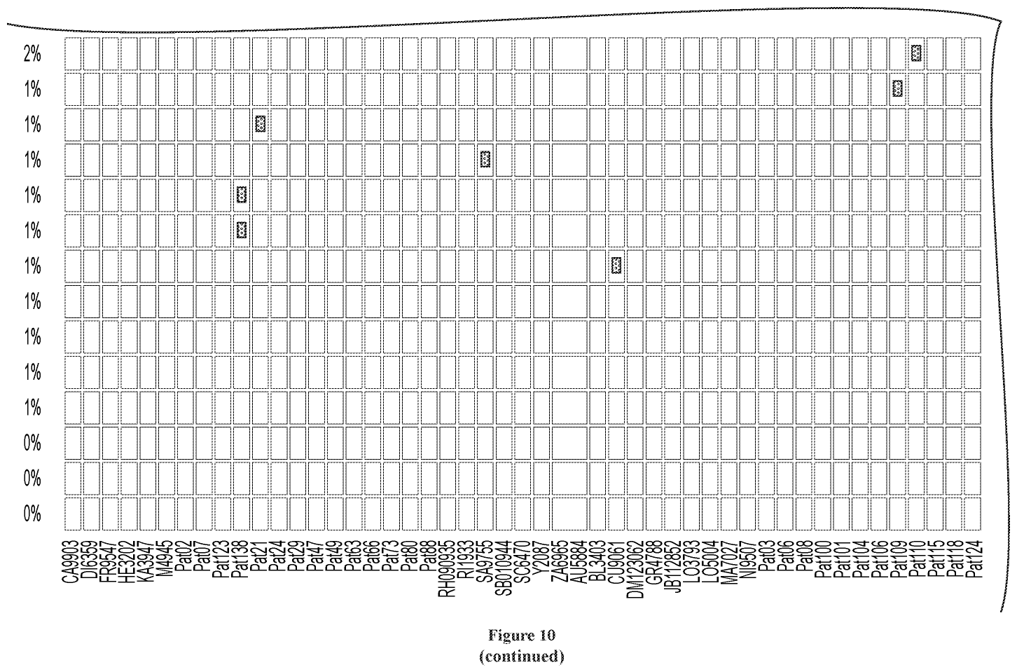

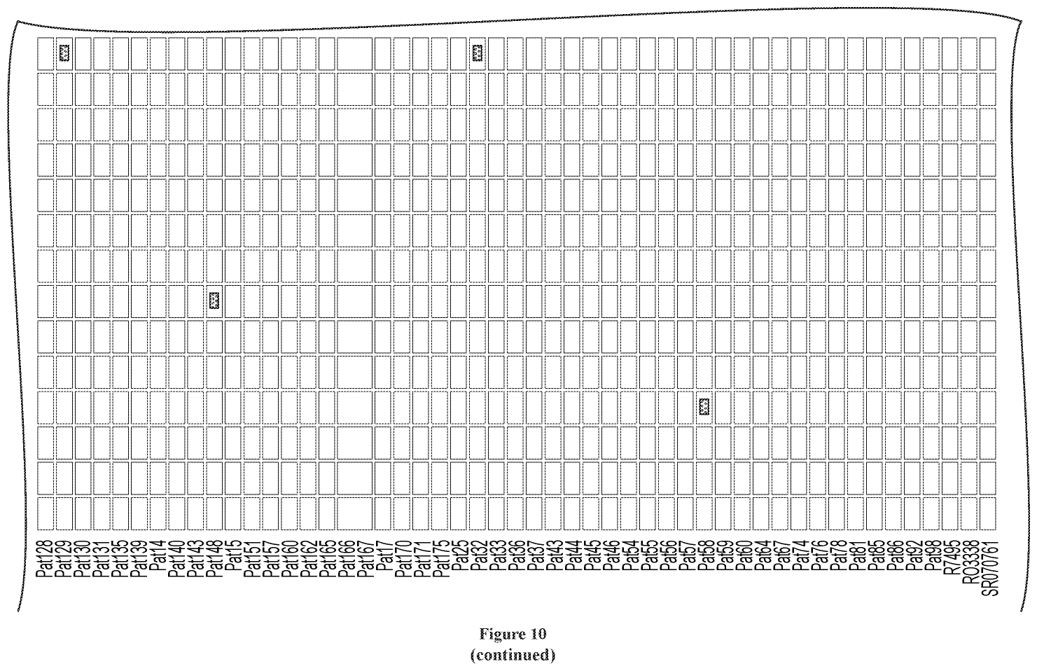

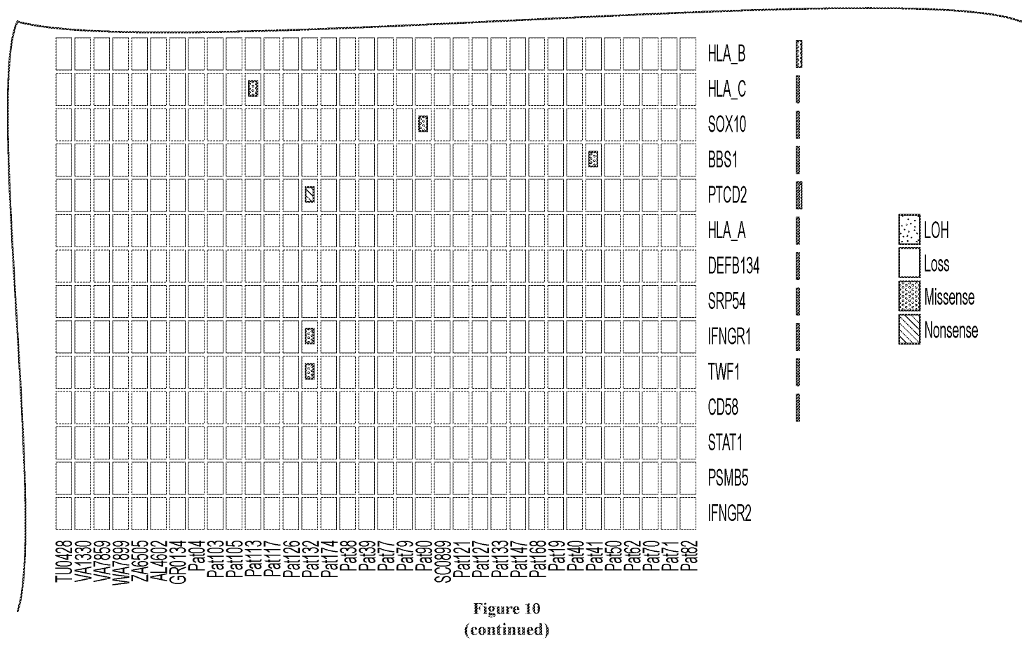

We examined all cohorts from FIG. 2 to investigate the combined effect of mean HED and TMB on response to ICI. First, we found that the effect of mean HED on survival after ICI (FIG. 3a) was independent of other clinical variables in multivariable Cox regression analysis (FIG. 9a), and that high HED did not co-occur with known mutations in genes that have been reported to impact response to ICI44-48 (FIG. 10). We found that the combined effect of high HED and high TMB on overall survival after ICI was stronger than the effect of either alone as evidenced by the reduction in hazard ratio when stratifying patients by both variables^{49,50} (FIG. 3a-c). This effect was also observed when analyzing only fully heterozygous patients (FIG. 3d-f, FIG. 9b). Furthermore, this result remained robust across a wide range of cutpoints for HED and TMB (FIG. 3g) used to stratify patients into groups for survival analysis. Interestingly, we found that high HED at HLA-A and HLA-B were each associated with improved response to ICI, when considering all patients or only fully heterozygous patients (FIG. 3h), suggesting that divergence at individual class I loci may differentially affect ICI efficacy. In support of these analyses, we also detected the effect of high mean HED on improved overall survival after ICI in an additional pan-cancer dataset of over a thousand patients (FIG. 15).

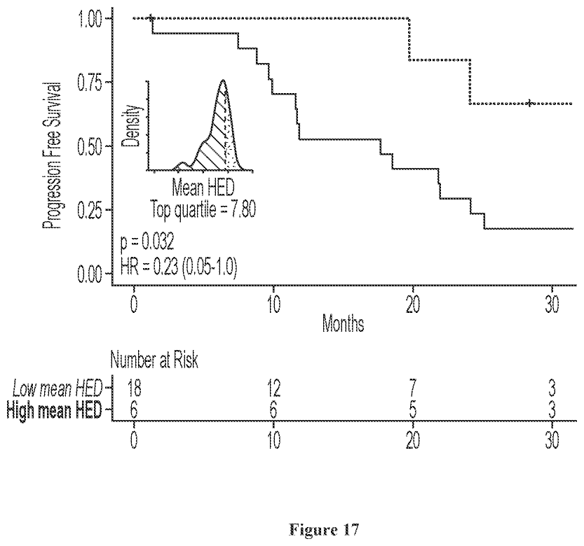

Further, we've determined that patients with renal cell carcinoma (RCC) with high mean HED demonstrate increased progression free survival after treatment with a combination therapy. Specifically, RCC patients treated with a combination of lenvatinib plus pembrolizumab showed increased progression free survival (FIG. 17 P-value=0.032; HR=0.23). The top quartile value was used as the cutoff to define high mean HED patients and low mean HED patients.

A possible explanation for the improved survival after ICI among patients who were fully heterozygous at HLA-I, and had high HED, is that higher HED may be associated with increased diversity of the neopeptide repertoire presented by HLA-I. Such an effect would lead to a higher probability of T-cell responses after administration of immunotherapy, due to the larger and more diverse immunopeptidome presented to cytotoxic T cells. This hypothesis is supported by the idea that pathogen-driven selection has maintained HLA-I polymorphism worldwide^{32,49,50}, and the idea that neoantigen recognition is driven in part by sequence similarity to pathogenic epitopes⁵¹⁻⁵³.

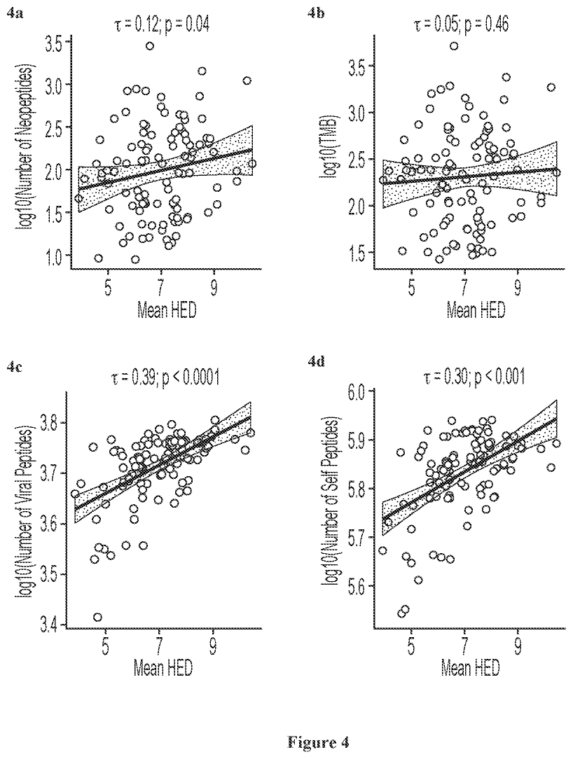

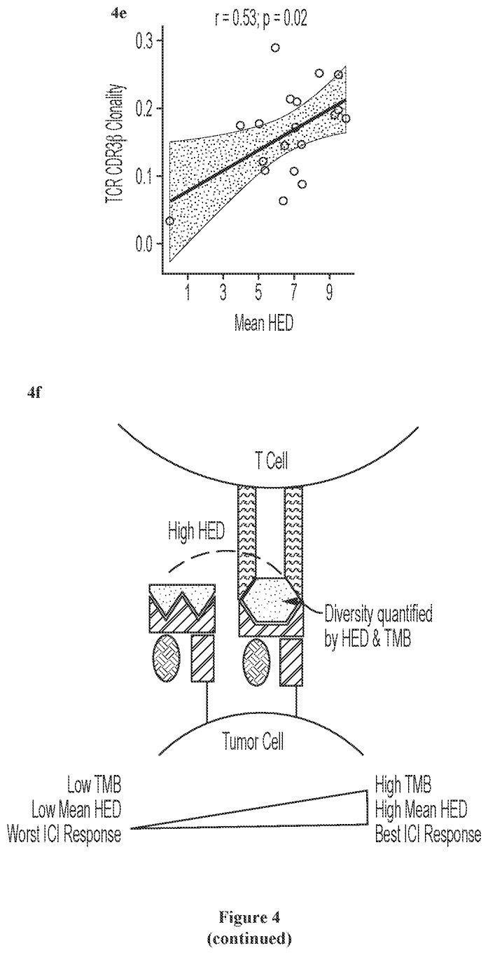

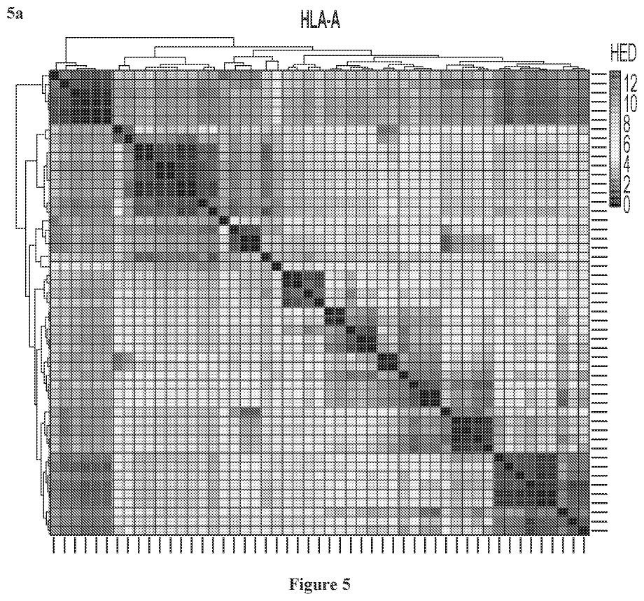

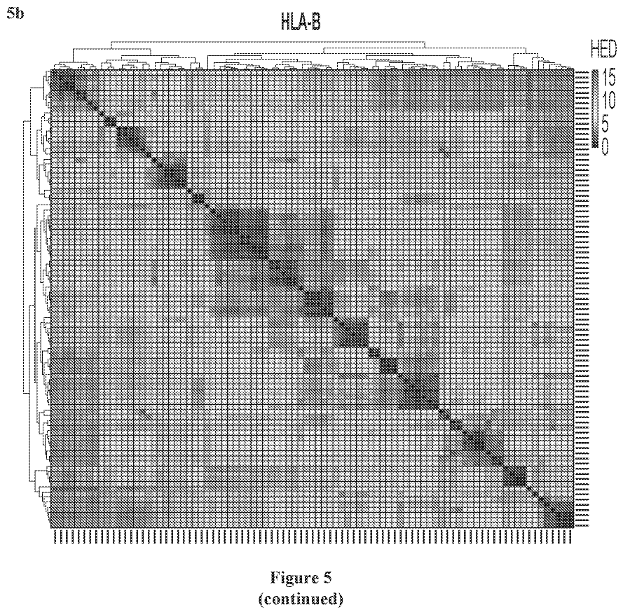

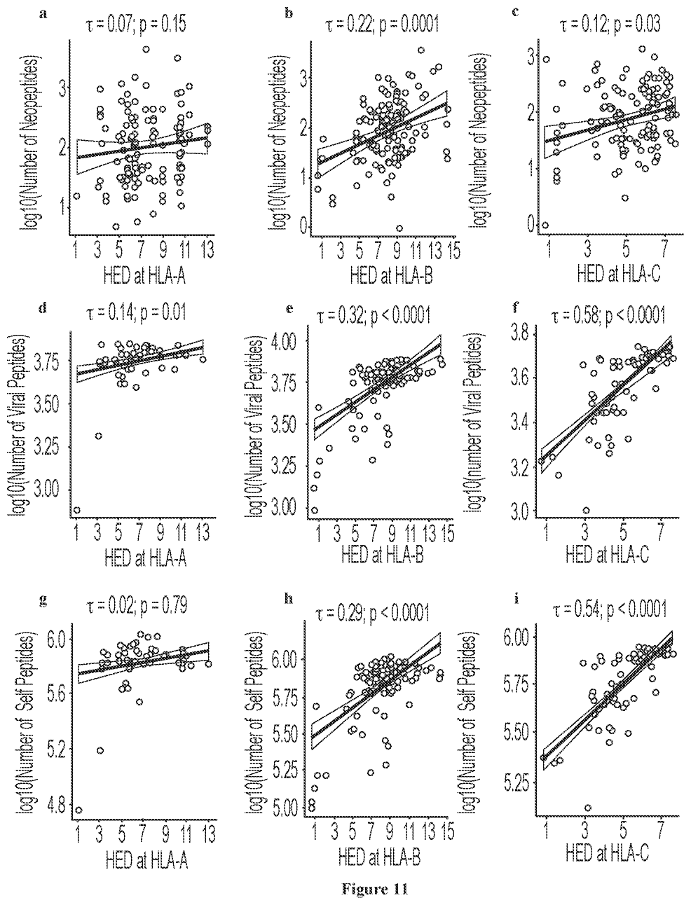

In an exploratory analysis limited to patients fully heterozygous at each locus, we found that the number of candidate neopeptides bound by heterozygous genotypes is correlated with mean HED (FIG. 4a). Moreover, mean HED was not correlated with tumor mutational burden (FIG. 4b), indicating that the diversity in HLA-I peptide-binding domains specifically reflects the diversity of neopeptide repertoire binding to the HLA-I molecules, rather than overall tumor mutation burden. We further detected associations between HED and diversity of the neopeptide repertoire at individual class loci (FIG. 11a-c). Consistent with these results, HED was also correlated with the abundance of viral peptides derived from a number of pathogens (FIG. 4c, FIG. 11).

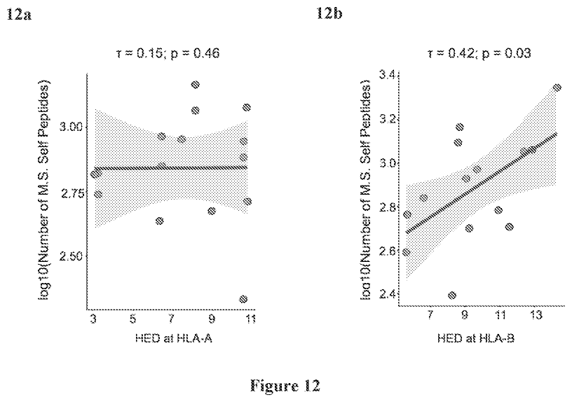

We next examined whether HED is also associated with total human self immunopeptidome diversity. We computationally generated all unique peptides of length nine from the entire human proteome to enable a common reference self-proteome across all patients, and performed HLA-I binding predictions. We found that HED was correlated with diversity of the predicted self immunopeptidome (FIG. 4c, FIG. 11d-f). We further determined HED in an independent cohort of 12 individuals for which naturally eluted peptide data were available⁵⁴, and observed an association between HED at HLA-B and self immunopeptidome diversity (FIG. 12), recapitulating our findings from peptide binding predictions. We also analyzed an additional dataset of mass spectrometry-derived peptidomes from mono-allelic cells48, which includes peptide data for 10 HLA-A and 6 HLA-B alleles. We computed HLA-I evolutionary divergences and the number of peptides bound by all possible pairs of HLA-A and HLA-B alleles (N=120), and found a significant negative correlation between HED and the overlap of peptides bound by both alleles of a given pair (FIG. 16a), which indicates that the more divergent the HLA-I alleles are, the more distinct the peptides they present. We also detected a similar negative correlation when considering HLA-A alleles alone (FIG. 16b), or HLA-B alleles alone (FIG. 16c). Furthermore, we found that HED was positively correlated with the abundance of peptides bound to pairs of alleles at each individual locus (FIG. 16 d-e). Altogether, these data suggest for the first time that increased sequence divergence of a HLA-I genotype is associated with increased diversity of self, tumor, and viral immunopeptidomes.

We further investigated whether HED could be associated with the clonality of the intratumoral TCR repertoire. We hypothesized that the association of high HED with a broader neopeptide repertoire would increase the probability of neoantigen recognition by tumor-infiltrating T-cells, and subsequently influence T cell clonal expansion. Accordingly, in a subset of patients treated with ICI therapy for whom next-generation deep sequencing of T cell receptor CDR3 regions (TCR-seq) were available55, we found a positive correlation between mean HED and clonality of TCR CDR3s (FIG. 4d). However, additional patient cohorts with whole-exome and TCR-sequencing will be required to confirm this result. Importantly, since TCRs interact with self-peptides presented by each individual's HLA-I molecules during thymic selection, HED may affect the diversity of the TCR repertoire of T cells in peripheral blood.

Taken together, these data show that HLA-I evolutionary divergence—as measured by sequence divergence between alleles of a HLA-I genotype—is associated with response to checkpoint blockade immunotherapy in patients treated for cancer, and the diversity of tumor, viral, and human immunopeptidomes. We note that HED can be reliably inferred from normal DNA sequencing. Furthermore, our results suggest that patients with both high TMB and high HED are most likely to benefit from ICI.

One explanation for these findings is that highly divergent HLA-I genotypes can present more diverse antigenic epitopes (FIG. 4e), which may lead to the expansion of T cell clones that facilitate superior tumor control. In such cases, it may be more challenging for a tumor clone to evade recognition by the host immune system during immunotherapy. Further studies may investigate the effect of HED on tumor evolution56 and specificity of the host T cell receptor repertoire⁵⁷.

### Example 2: Methods

### Description of Patient Cohorts

We used four previously published cohorts of patients with late-stage melanoma and non-small cell lung cancer (NSCLC) treated with anti-CTLA-4, or PD-1/PD-L1 blockade^{5,8,14,43}. Ten patients from the Van Allen et al. cohort were excluded, since they achieved long-term survival after anti-CTLA-4 treatment with early tumor progression⁸. The NSCLC data are from patients with metastatic disease treated mainly with anti-PD-1 monotherapy. The patients are from a prospective trial that we reported previously⁵ and from New York-Presbyterian/Columbia University Medical Center¹⁴. From these NSCLC cohorts, for the analyses involving combination of HED and TMB (FIG. 3), only patients with exome sequencing data were included, since mutation data were not available for 66 patients with NSCLC. For the analyses involving HED only (FIG. 2), all NSCLC patients were included, since HLA types were available for all patients. All patients were treated under institutional review approved prospective protocols. Clinical characteristics of patient cohorts are provided in the original studies. The TCGA exome data for the patients with melanoma (N=457) and lung cancer (N=545) was obtained from The Cancer Genome Atlas (TCGA) (http://cancergenome.nih.gov).

### Overall Survival and Clinical Response

Overall survival was defined as the length of time from treatment start to time to event (survival or censor). Response data was available for some cohorts^{5,8,14}; clinical benefit was defined as complete response, partial response, or stable disease as indicated in previous studies^{5,8,14}. No clinical benefit was defined as progressive disease. All clinical data, including overall survival and clinical response data, were obtained from the original studies. Clinical data for the TCGA patients with melanoma and non-small cell lung cancer were obtained through the TCGA data portal.

### HLA Class I Genotyping

We performed HLA-I genotyping as described previously¹⁴. Briefly, we performed high-resolution HLA class I genotyping from germline normal DNA exome sequencing data directly or using a clinically validated HLA typing assay (LabCorp). Patient exome data or targeted gene panels were obtained and the well-validated tool Polysolver was used to identify HLA class I alleles with default parameter settings⁵⁸. For the 67 patients with NSCLC with no available exome sequencing data, HLA class I typing was done at LabCorp. For quality assurance of HLA-I genotyping using MSK-IMPACT (CLIA-approved hybridization-capture based assay) with melanoma samples from anti-PD1-treated patients, we compared HLA class I typing by Polysolver between 37 samples that we sequenced with MSK-IMPACT and whole exome. The MSK-IMPACT panel successfully captured HLA-A, -B, and -C reads and validation was previously performed¹⁴. The overall concordance of class I typing between the MSK-IMPACT samples and their matched WES samples was 96%. To make sure that HLA class I genes have adequate coverage in MSK-IMPACT bam files, we also applied bedtools multicov tool (htip://bedtools.readthedocs.io/en/latest/content/tools/multicov.html), which reports the count of alignments from multiple position-sorted and indexed BAM files that overlap with targets intervals in a BED format. Only high quality reads were counted and only samples with sufficient coverage were used. Patients were considered fully heterozygous at HLA-I if they have 6 different HLA-I alleles.

### Calculation of Patient HLA-I Evolutionary Divergence (HED)

HLA-I genotype divergence was calculated as described in Pierini and Lenz³². Briefly, we first extracted the protein sequence of exons 2 and 3 of each allele of each patient's HLA-I genotype, which correspond to the peptide-binding domains. Protein sequences were obtained from the IMGT/HLA database⁵⁹, and exons coding for the variable peptide-binding domains were selected following the annotation obtained from Ensembl database⁶⁰. Sequence divergences between allele sequences were calculated using the Grantham distance metric³⁴, as implemented in Pierini and Lenz³². The Grantham distance is a quantitative pairwise distance in which the physiochemical properties of amino acids, and hence the functional similarity between sequences are considered³⁴. Given a particular HLA-I locus with two alleles, the sequences of the peptide-binding domains of each allele are aligned⁶¹, and the Grantham distance is calculated as the sum of amino acid differences (taking into account the biochemical composition, polarity, and volume of each amino acid) along the sequences of the peptide-binding domains, following the formula by R. Grantham³⁴:

Grantham Distance=ΣDᵢⱼ=Σ[α(cᵢ−cⱼ)²+β(pᵢ−pⱼ)²+γ(vᵢ−vⱼ)²]^{1/2} (1)

where i and j are the two homologous amino acids at a given position in the alignment, c, p, and v represent composition, polarity, and volume of the amino acids respectively, and α, β, and γ are constants; all values are taken from the original study³⁴. The final Grantham distance is calculated by normalizing the value from (1) by the length of the alignment between the peptide-binding domains of a particular HLA-I genotype's two alleles. A prior analysis of multiple common sequence divergence measures showed that the correlation of Grantham distance with the number of peptides bound by both alleles of a heterozygous genotype exceeded that of the other distance measures³². Patient mean HED was calculated as the mean of divergences at HLA-A, HLA-B, and HLA-C.

### Tumor Mutational Analysis Pipeline

For cohorts which received whole exome sequencing, reads in FASTQ format were aligned to the reference human genome GRCh37 using the Burrows-Wheeler aligner (BWA; v0.7.10)⁶². Local realignment was performed using the Genome Analysis Toolkit (GATK 3.7)⁶³. Duplicate reads were removed using Picard version 2.13. To identify somatic single nucleotide variants (SNVs), we used a validated pipeline that integrates mutation calls from four different mutation callers: MuTect 1.1.7, Strelka 1.0.15, SomaticSniper 1.0.4, and Varscan 2.4.3⁶⁴⁻⁶⁷. SNVs with an alternative allele read count of less than 4, total coverage of less then 10 or with corresponding normal coverage of less than 7 reads were filtered out.

### Computational Identification of HLA-I Restricted Neopeptides

Each non-synonymous SNV was translated into a 17-mer peptide sequence, centered on the mutated amino acid. Adjacent SNVs were corrected using MAC⁶⁸. Subsequently, the 17-mer was then used to create 9-mers via a sliding window approach for determination of HLA-I binding predictions for neopeptides using NetMHCpan-4.0⁶⁹. All peptides with a rank <2% were considered for further analyses.

Computational Identification of HLA-I Restricted Peptides from the Human Proteome and Viral Antigens

We identified peptides from the entire human proteome that bind to patient-specific HLA-I alleles. The human peptidome was downloaded from Ensemb1⁶⁰ (ftp://ftp.ensembl.org/pub/grch37/update/fasta/homo_sapiens/pep//Homo_sapiens.GRCh37. pep.all.fa). Only sequences annotated as gene_biotype:protein_coding and transcript biotype:protein_coding were kept. Transcripts with identical sequences were de-duplicated. The resulting FASTA file was submitted to NetMHCpan-4.0⁶⁹ to determine HLA-I binding predictions. All peptides from the human proteome with a rank <2% were considered for further analyses. For the peptide-divergence correlation analyses in FIG. 12, we used self peptides identified via mass spectrometry and HLA-I genotypes from Pearson et al⁵⁴. All correlation analyses were limited to peptides of length 9. We additionally generated predicted viral peptides from a number of antigens.

### TCR β-Chain Sequencing and Analysis

We employed next-generation sequencing of TCR β-chain complementarity determining regions (CDR3s) (TCR-seq) (Adaptive Biotechnologies)^{70,71} from a subset of tumor samples collected pre-therapy from responders (CR/PR/SD) in the Riaz et al cohort⁵⁵. We subsequently calculated the clonality of the TCR CDR3 repertoire, defined as the complement of evenness (i.e., 1−evenness). Evenness is defined as the observed Shannon entropy (H) divided by the maximum possible H, given the number of unique elements in a population. Data for individual TCR sequences, were obtained from Adaptive Biotechnologies for customized analysis of T cell repertoire. Correlation analyses were performed using Pearson's r.

### Genomic Oncoprint

The oncoprint displays mutated genes that have been reported to impact response to ICI. The genes in the IFNG gene cluster on 9p are: IFNA1, IFNA10, IFNA13, IFNA14, IFNA16, IFNA17, IFNA2, IFNA21, IFNA22P, IFNA4, IFNA, IFNA6, IFNA7, IFNA8, IFNB1, IFNE, IFNW1. The Loss events were identified in the following manner: 1. Rounded FACETS ploidy value to the nearest whole number; 2. Used rounded ploidy value to correct Total Copy Number (tcn.em) with: Corrected_TCN=tcn.em—rounded_ploidy; 3. If Corrected_TCN<=−1, then marked as a “Loss” Event. Note: this computation was performed for each FACETS' segment on chromosome 9 and was assigned to individual genes with coordinates within the FACETS segment. Homozygous loss events were identified If tcn.em=0 (did not use ploidy-corrected TCN). All losses were manually verified. For assessing loss of heterozygosity (LOH) of HLA class I, copy number variation analysis was performed using FACETS 0.5.6 to determine allele specific copy number⁷². Segments within the chromosome 6p locus were identified containing the HLA-A, HLA-B and HLA-C loci. Loss of heterozygosity (LOH) was defined as a minor allele copy number estimate of 0 for any of the HLA-I loci using the expectation-maximization mode1⁷².

### Correlation Analyses

We limited all HLA-I evolutionary divergence (HED)-peptide correlation analyses to patients heterozygous at each locus only. For FIGS. 4a and c, the y-axis shows the mean number of neo- or self peptides bound uniquely to each allele for each of HLA-A, B, and C. For the analysis shown in FIG. 11f, two patients had an HLA-C genotype (C*03:03,C*03:04) that bound a total of 0 neopeptides. These patients were excluded from the plot for visualization purposes only. The correlation displayed in FIG. 11f is significant regardless of whether these patients are included. We used the nonparametric Kendall correlation as shown in Pierini and Lenz³², since parameters were not normally distributed. For FIG. 4a and FIG. 11a-11c, we used one-sided p-values—given the prior association of genotype divergence with diversity of nonself pathogenic peptides shown by Pierini and Lenz³²; we hypothesized that a similar association would be observed for the neopeptide correlations. For FIG. 4c and FIG. 11d-11f, we had no prior hypothesis regarding the direction of the association between genotype divergence and diversity of self-peptides from the human proteome; thus, two-sided p-values were used.

### Statistical Analyses

Comparisons of HED distributions across individual HLA-I loci were calculated using the Mann-Whitney test. Survival analyses were performed using the Kaplan-Meier estimator. All cutoffs for germline HLA-I evolutionary divergence (HED) and tumor mutational burden (TMB) were determined using the top quartile. For analyses in FIG. 3 combining cohorts with whole-exome and targeted panel sequencing, the TMB of the whole-exome cohorts was divided by 30 to normalize per megabase⁴³. We performed the survival analysis in FIG. 6a using the mean of divergences at HLA-A, B, and C as well as the sum, median, and geometric mean. Results were similar across all metrics used (Table 1). Table 1 shows survival analysis from FIG. 10a using different metrics for aggregating divergence across HLA-A, B, and C. Table shows results from log-rank test for data from FIG. 6a when using various metrics (mean, sum, median, or geometric mean) to aggregate HLA-I evolutionary divergences across HLA-A, B, and C. Table 1 shows that results from FIG. 6a do not depend on the metric chosen.

TABLE 1

| | Log-rank | Hazard | 95% Confidence |
|---|---|---|---|
| Metric | p-value | Ratio | Interval |
| Mean | 0.0072 | 0.47 | 0.26-0.82 |
| Sum | 0.0072 | 0.47 | 0.26-0.82 |
| Median | 0.067 | 0.6 | 0.35-1.04 |
| Geometric | 0.015 | 0.5 | 0.29-0.89 |
| Mean | | | |

For the analysis in FIG. 13, each mean HED value in the dataset was used as a cutpoint for high mean HED in survival analysis, and hazard ratios were calculated from univariable cox regression. These hazard ratios were plotted against all mean HED values. For the analysis in FIG. 3g, we used each value of mean HED in the data as a cutpoint for high HED, and did the same for TMB. When combining mean HED and TMB, patients were in the high group if their mean HED and TMB were both greater than the cutpoints for mean HED and TMB, and in the low group if both variables were less than their respective cutpoint. For all multivariable analyses, P-values and hazard ratios were calculated using the Cox regression. For all survival analyses, P-values were calculated using the log-rank test, and hazard ratios were calculated using the univariable Cox regression. For the analyses of clinical response data, P-values and odds ratios (OR) were calculated using Fisher's exact test (two-sided). All survival and correlation analyses were performed in the R Statistical Computing Environment version 3.5.0 (http://www.r-project.org).

Data availability

The data from prior studies are available at the following accession numbers: dbGAP, phs000452.v2.p1; dbGAP, phs000980.v1.p1; SRA, PRJNA419415, PRJNA419422, and PRJNA419530; TCGA data are available from the cBioPortal for Cancer Genomics, cbioportal.org/msk-impact, cancergenome.nih.gov.

### REFERENCES

1. Callahan, M. K., Postow, M. A. & Wolchok, J. D. Targeting T Cell Co-receptors for Cancer Therapy. Immunity 44, 1069-1078 (2016).
2. McGranahan, N., et al. Clonal neoantigens elicit T cell immunoreactivity and sensitivity to immune checkpoint blockade. Science 351, 1463-1469 (2016).
3. Samstein, R. M., et al. Tumor mutational load predicts survival after immunotherapy across multiple cancer types. Nat Genet (2019).
4. Snyder, A., et al. Genetic basis for clinical response to CTLA-4 blockade in melanoma. N Engl J Med 371, 2189-2199 (2014).
5. Rizvi, N. A., et al. Cancer immunology. Mutational landscape determines sensitivity to PD-1 blockade in non-small cell lung cancer. Science 348, 124-128 (2015).
6. Yarchoan, M., Hopkins, A. & Jaffee, E. M. Tumor Mutational Burden and Response Rate to PD-1 Inhibition. N Engl J Med 377, 2500-2501 (2017).
7. Goodman, A. M., et al. Tumor Mutational Burden as an Independent Predictor of Response to Immunotherapy in Diverse Cancers. Mol Cancer Ther 16, 2598-2608 (2017).
8. Van Allen, E. M., et al. Genomic correlates of response to CTLA-4 blockade in metastatic melanoma. Science 350, 207-211 (2015).
9. Le, D. T., et al. Mismatch-repair deficiency predicts response of solid tumors to PD-1 blockade. Science (2017).
10. Tran, E., et al. Immunogenicity of somatic mutations in human gastrointestinal cancers. Science 350, 1387-1390 (2015).
11. Tran, E., et al. T-Cell Transfer Therapy Targeting Mutant KRAS in Cancer. N Engl J Med 375, 2255-2262 (2016).
12. van Rooij, N., et al. Tumor exome analysis reveals neoantigen-specific T-cell reactivity in an ipilimumab-responsive melanoma. J Clin Oncol 31, e439-442 (2013).
13. Schumacher, T. N. & Schreiber, R. D. Neoantigens in cancer immunotherapy. Science 348, 69-74 (2015).
14. Chowell, D., et al. Patient HLA class I genotype influences cancer response to checkpoint blockade immunotherapy. Science 359, 582-587 (2018).
15. Carrington, M., et al. HLA and HIV-1: heterozygote advantage and B*35-Cw*04 disadvantage. Science 283, 1748-1752 (1999).
16. Penn, D. J., Damjanovich, K. & Potts, W. K. MHC heterozygosity confers a selective advantage against multiple-strain infections. Proc Natl Acad Sci USA 99, 11260-11264 (2002).
17. Thursz, M. R., Thomas, H. C., Greenwood, B. M. & Hill, A. V. Heterozygote advantage for HLA class-II type in hepatitis B virus infection. Nat Genet 17, 11-12 (1997).
18. Doherty, P. C. & Zinkernagel, R. M. A biological role for the major histocompatibility antigens. Lancet 1, 1406-1409 (1975).
19. Doherty, P. C. & Zinkernagel, R. M. Enhanced Immunological Surveillance in Mice Heterozygous at H-2 Gene Complex. Nature 256, 50-52 (1975).
20. Parham, P. & Ohta, T. Population biology of antigen presentation by MHC class I molecules. Science 272, 67-74 (1996).
21. Hughes, A. L. & Yeager, M. Natural selection at major histocompatibility complex loci of vertebrates. Annu Rev Genet 32, 415-435 (1998).
22. Parham, P. Function and polymorphism of human leukocyte antigen-A,B,C molecules. Am J Med 85, 2-5 (1988).
23. Robinson, J., et al. Distinguishing functional polymorphism from random variation in the sequences of >10,000 HLA-A, —B and -C alleles. Plos Genetics 13(2017).
24. Hughes, A. L. & Nei, M. Pattern of Nucleotide Substitution at Major Histocompatibility Complex Class-I Loci Reveals Overdominant Selection. Nature 335, 167-170 (1988).
25. Gfeller, D. & Bassani-Sternberg, M. Predicting Antigen Presentation-What Could We Learn From a Million Peptides? Front Immunol 9, 1716 (2018).
26. La Gruta, N. L., Gras, S., Daley, S. R., Thomas, P. G. & Rossjohn, J. Understanding the drivers of MHC restriction of T cell receptors. Nature Reviews Immunology 18, 467-478 (2018).
27. Rammensee, H. G., Friede, T. & Stevanoviic, S. MHC ligands and peptide motifs: first listing. Immunogenetics 41, 178-228 (1995).
28. Sette, A. & Sidney, J. Nine major HLA class I supertypes account for the vast preponderance of HLA-A and -B polymorphism. Immunogenetics 50, 201-212 (1999).
29. Paul, S., et al. HLA class I alleles are associated with peptide-binding repertoires of different size, affinity, and immunogenicity. J Immunol 191, 5831-5839 (2013).
30. Wakeland, E. K., et al. Ancestral polymorphisms of MHC class II genes: divergent allele advantage. Immunol Res 9, 115-122 (1990).
31. Potts, W. K. & Wakeland, E. K. Evolution of Diversity at the Major Histocompatibility Complex. Trends Ecol Evol 5, 181-187 (1990).
32. Pierini, F. & Lenz, T. L. Divergent allele advantage at human MHC genes: signatures of past and ongoing selection. Mol Biol Evol (2018).
33. Lenz, T. L. Computational prediction of MHC II-antigen binding supports divergent allele advantage and explains trans-species polymorphism. Evolution 65, 2380-2390 (2011).
34. Grantham, R. Amino acid difference formula to help explain protein evolution. Science 185, 862-864 (1974).
35. McKenzie, L. M., Pecon-Slattery, J., Carrington, M. & O'Brien, S. J. Taxonomic hierarchy of HLA class I allele sequences. Genes Immun 1, 120-129 (1999).
36. Grueber, C. E., Wallis, G. P. & Jamieson, I. G. Episodic positive selection in the evolution of avian toll-like receptor innate immunity genes. PLoS One 9, e89632 (2014).
37. Subramanian, S. & Kumar, S. Evolutionary anatomies of positions and types of disease-associated and neutral amino acid mutations in the human genome. BMC genomics 7, 306 (2006).
38. Wain, L. V., et al. Adaptation of HIV-1 to its human host. Mol Biol Evol 24, 1853-1860 (2007).
39. Feng, S., et al. The Genomic Footprints of the Fall and Recovery of the Crested Ibis. Curr Bio 129, 340-349 e347 (2019).
40. International Wheat Genome Sequencing, C. A chromosome-based draft sequence of the hexaploid bread wheat (Triticum aestivum) genome. Science 345, 1251788 (2014).
41. Rentoft, M., et al. Heterozygous colon cancer-associated mutations of SAMHD1 have functional significance. Proc Natl Acad Sci USA 113, 4723-4728 (2016).
42. Sundaram, L., et al. Predicting the clinical impact of human mutation with deep neural networks. Nat Genet 50, 1161-1170 (2018).
43. Zehir, A., et al. Mutational landscape of metastatic cancer revealed from prospective clinical sequencing of 10,000 patients. Nat Med 23, 703-713 (2017).
44. Gao, J., et al. Loss of IFN-gamma Pathway Genes in Tumor Cells as a Mechanism of Resistance to Anti-CTLA-4 Therapy. Cell 167, 397-404 e399 (2016).
45. Patel, S. J., et al. Identification of essential genes for cancer immunotherapy. Nature 548, 537-542 (2017).
46. Zaretsky, J. M., et al. Mutations Associated with Acquired Resistance to PD-1 Blockade in Melanoma. N Engl J Med 375, 819-829 (2016).
47. Zhao, F., et al. Melanoma Lesions Independently Acquire T-cell Resistance during Metastatic Latency. Cancer Res 76, 4347-4358 (2016).
48. Sade-Feldman, M., et al. Resistance to checkpoint blockade therapy through inactivation of antigen presentation. Nature Communications 8(2017).
49. Prugnolle, F., et al. Pathogen-driven selection and worldwide HLA class I diversity. Curr Bio 115, 1022-1027 (2005).
50. Buhler, S., Nunes, J. M. & Sanchez-Mazas, A. HLA class I molecular variation and peptide-binding properties suggest a model of joint divergent asymmetric selection. Immunogenetics 68, 401-416 (2016).
51. Balachandran, V. P., et al. Identification of unique neoantigen qualities in long-term survivors of pancreatic cancer. Nature 551, 512-516 (2017).
52. Luksza, M., et al. A neoantigen fitness model predicts tumour response to checkpoint blockade immunotherapy. Nature 551, 517-520 (2017).
53. Kim, S., et al. Neopepsee: accurate genome-level prediction of neoantigens by harnessing sequence and amino acid immunogenicity information. Ann Oncol (2018).
54. Pearson, H., et al. MHC class I-associated peptides derive from selective regions of the human genome. Journal of Clinical Investigation 126, 4690-4701 (2016).
55. Riaz, N., et al. Tumor and Microenvironment Evolution during Immunotherapy with Nivolumab. Cell (2017).
56. Greaves, M. & Maley, C. C. Clonal evolution in cancer. Nature 481, 306-313 (2012).
57. Sharon, E., et al. Genetic variation in MHC proteins is associated with T cell receptor expression biases. Nat Genet 48, 995-1002 (2016).
58. Shukla, S. A., et al. Comprehensive analysis of cancer-associated somatic mutations in class I HLA genes. Nat Biotechnol 33, 1152-1158 (2015).
59. Robinson, J., et al. The IPD and IMGT/HLA database: allele variant databases. Nucleic Acids Research 43, D423-D431 (2015).
60. Zerbino, D. R., et al. Ensembl 2018. Nucleic Acids Res 46, D754-D761 (2018).
61. Edgar, R. C. MUSCLE: multiple sequence alignment with high accuracy and high throughput. Nucleic Acids Res 32, 1792-1797 (2004).
62. Li, H. & Durbin, R. Fast and accurate short read alignment with Burrows-Wheeler transform. Bioinformatics 25, 1754-1760 (2009).
63. McKenna, A., et al. The Genome Analysis Toolkit: a MapReduce framework for analyzing next-generation DNA sequencing data. Genome Res 20, 1297-1303 (2010).
64. Cibulskis, K., et al. Sensitive detection of somatic point mutations in impure and heterogeneous cancer samples. Nature Biotechnology 31, 213-219 (2013).
65. Koboldt, D. C., et al. VarScan 2: Somatic mutation and copy number alteration discovery in cancer by exome sequencing. Genome Research 22, 568-576 (2012).
66. Larson, D. E., et al. SomaticSniper: identification of somatic point mutations in whole genome sequencing data. Bioinformatics 28, 311-317 (2012).
67. Saunders, C. T., et al. Strelka: accurate somatic small-variant calling from sequenced tumor-normal sample pairs. Bioinformatics 28, 1811-1817 (2012).
68. Wei, L., et al. MAC: identifying and correcting annotation for multi-nucleotide variations. BMC genomics 16, 569 (2015).
69. Jurtz, V., et al. NetMHCpan-4.0: Improved Peptide-MHC Class I Interaction Predictions Integrating Eluted Ligand and Peptide Binding Affinity Data. J Immuno 1199, 3360-3368 (2017).
70. Carlson, C. S., et al. Using synthetic templates to design an unbiased multiplex PCR assay. Nature Communications 4(2013).
71. Robins, H. S., et al. Comprehensive assessment of T-cell receptor beta-chain diversity in alphabeta T cells. Blood 114, 4099-4107 (2009).
72. Shen, R. L. & Seshan, V. E. FACETS: allele-specific copy number and clonal heterogeneity analysis tool for high-throughput DNA sequencing. Nucleic Acids Research 44(2016).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method comprising steps of:
administering immune checkpoint inhibitor therapy to a subject suffering from cancer who has a high HLA-I evolutionary divergence (HED).

2. A method comprising steps of:
determining a HLA-I evolutionary divergence (HED) of a subject suffering from cancer;
identifying a subject with a high HED as a candidate for treatment with an immunotherapy.

3. A method of treating a subject suffering from cancer comprising:
determining that the subject has a high HLA-I evolutionary divergence (HED);
administering immunotherapy.

4. A method of determining if a subject suffering from cancer will respond to immunotherapy comprising:
determining the subjects HLA-I evolutionary divergence (HED);
wherein a high HED indicates a subject will be more responsive to immunotherapy.

5. The methods of any one of claims 1 to 4 wherein the HED is determined by quantifying the sequence divergence between HLA class I alleles.

6. The methods of any one of claims 1 to 4 wherein the HED is determined by quantifying the sequence divergence between HLA class I alleles through measurement of the Grantham distance.

7. The methods of any one of claims 1 to 4 wherein HED is determined as the mean evolutionary divergence of the HLA-A; HLA-B; and HLA-C genes.

8. The methods of any one of claims 1 to 7 wherein the cancer is a solid tumor

9. The methods of any one of claims 1 to 7 wherein the cancer is melanoma or non-small cell lung cancer.

10. The methods of any one of claims 1 to 7 wherein the immunotherapy is or comprises administration of an immune checkpoint modulator.

11. The methods of any one of claims 1 to 7 wherein the immunotherapy is or comprises administration of an antibody agent.

12. The methods of any one of claims 1 to 7 wherein the immunotherapy is or comprises administration of a monoclonal antibody.

13. The methods of any one of claims 1 to 7 wherein the immunotherapy is or comprises administration of one or more of PD-1 or PD-L1 blockade therapies.

14. The methods of any one of claims 1 to 7 wherein the immunotherapy is or comprises administration of one or more of CTLA-4 blockade therapies.

15. The methods of any one of claims 1 to 7 wherein the immunotherapy is a combination of one or more of PD-1 blockade therapy and CTLA-4 blockade therapy.

16. The methods of any one of claims 1 to 7 wherein the immunotherapy is selected from the group comprising of atezolizumab, avelumab, durvalumab, ipilimumab, nivolumab, pembrolizumab, or tremelimumab, and combinations therein.

17. The methods of any one of claims 1 to 7 wherein the subject is fully heterozygous for HLA-I.

18. The methods of any one of claims 1 to 7, wherein the subject further has high tumor mutational burden.

19. In a method of administering immune checkpoint inhibitor therapy to treat cancer, the improvement that comprises:
administering the immune checkpoint inhibitor therapy to subjects whose HLA-I evolutionary divergence (HED) is high.

20. In a method of administering immune checkpoint inhibitor therapy to treat cancer, the improvement that comprises:
selecting a subject whose mean HLA-I evolutionary divergence (HED) is high to receive the therapy; and
administering the therapy to the subject.

21. A method of assessing an immune checkpoint inhibitor therapy regimen by:
determining a degree of effectiveness of the regimen relative to HED.
